(19) United States
(12) Patent Application Publication
(10) Pub. No.: US 2015/0294463 A1
(43) Pub. Date: Oct. 15, 2015

(54) IMAGE PROCESSING DEVICE, ENDOSCOPE APPARATUS, IMAGE PROCESSING METHOD, AND INFORMATION STORAGE DEVICE

(71) Applicant: OLYMPUS CORPORATION, Tokyo (JP)

(72) Inventor: Jumpei TAKAHASHI, Tokyo (JP)

(73) Assignee: OLYMPUS CORPORATION, Tokyo (JP)

(21) Appl. No.: 14/728,067

(22) Filed: Jun. 2, 2015

Related U.S. Application Data
(63) Continuation of application No. PCT/JP2013/075626, filed on Sep. 24, 2013.

(30) Foreign Application Priority Data
Jan. 28, 2013 (JP) 2013-012816

(51) Int. Cl. G06T 7/00; A61B 1/04; A61B 1/00; H04N 9/04; H04N 7/18

(52) U.S. Cl. CPC G06T 7/0012; H04N 9/045; H04N 7/183; A61B 1/00193; A61B 1/04; H04N 2005/2255

(57) ABSTRACT

An image processing device includes an image acquisition section, a distance information acquisition section, a concavity-convexity information acquisition section, a determination section that determines whether or not to exclude or reduce extracted concavity-convexity information corresponding to each given area of a captured image, and a concavity-convexity information correction section that excludes the extracted concavity-convexity information corresponding to the given area for which the determination section has determined to exclude the extracted concavity-convexity information, or reduces the degree of concavities and convexities represented by the extracted concavity-convexity information corresponding to the given area for which the determination section has determined to reduce the extracted concavity-convexity information. The concavity-convexity information acquisition section excludes a structure that is more global than a desired concavity-convexity part from the distance information based on known characteristic information to extract information about the desired concavity-convexity part as the extracted concavity-convexity information.

### CROSS REFERENCE TO RELATED APPLICATION

This application is a continuation of International Patent Application No. PCT/JP2013/075626, having an international filing date of Sep. 24, 2013, which designated the United States, the entirety of which is incorporated herein by reference. Japanese Patent Application No. 2013-012816 filed on Jan. 28, 2013 is also incorporated herein by reference in its entirety.

### BACKGROUND

The present invention relates to an image processing device, an endoscope apparatus, an image processing method, an information storage device, and the like.

When observing tissue using an endoscope apparatus, and making a diagnosis, a method has been widely used that determines whether or not an early lesion has occurred by observing tissue as to the presence or absence of minute concavities and convexities (concavity-convexity parts). When using an industrial endoscope apparatus instead of a medical endoscope apparatus, it is useful to observe the object (e.g., the surface of the object) as to the presence or absence of a concavity-convexity structure in order to detect whether or not a crack has occurred in the inner side of a pipe that is difficult to directly observe with the naked eye, for example. It is also normally useful to detect the presence or absence of a concavity-convexity structure from the processing target image (object) when using an image processing device other than an endoscope apparatus.

For example, a process that enhances a concavity-convexity structure may be performed as a process that utilizes the concavity-convexity structure of the object. For example, a method that performs image processing that enhances a specific spatial frequency, and the method disclosed in JP-A-2003-88498, have been known as a method that enhances a structure (e.g., a concavity-convexity structure such as a groove) within the captured image by image processing. A method that effects some change in the object (e.g., dye spraying), and captures the object has also been known.

JP-A-2003-88498 discloses a method that enhances a concavity-convexity structure by comparing the luminance level of an attention pixel in a locally extracted area with the luminance level of its peripheral pixel, and coloring the attention area when the attention area is darker than the peripheral area.

### SUMMARY

According to one aspect of the invention, there is provided an image processing device comprising:

an image acquisition section that acquires a captured image that includes an image of an object;

a distance information acquisition section that acquires distance information based on a distance from an imaging section to the object when the imaging section captured the captured image;

a concavity-convexity information acquisition section that acquires concavity-convexity information about the object based on the distance information as extracted concavity-convexity information;

a determination section that determines whether or not to exclude or reduce the extracted concavity-convexity information corresponding to each given area of the captured image; and

a concavity-convexity information correction section that excludes the extracted concavity-convexity information corresponding to the given area for which the determination section has determined to exclude the extracted concavity-convexity information, or reduces a degree of concavities and convexities represented by the extracted concavity-convexity information corresponding to the given area for which the determination section has determined to reduce the extracted concavity-convexity information,

the concavity-convexity information acquisition section excluding a structure that is more global than a desired concavity-convexity part from the distance information based on known characteristic information to extract information about the desired concavity-convexity part as the extracted concavity-convexity information, the known characteristic information being information that represents known characteristics relating to a structure of the object.

According to another aspect of the invention, there is provided an endoscope apparatus comprising the image processing device.

According to another aspect of the invention, there is provided an image processing method comprising:

acquiring a captured image that includes an image of an object;

acquiring distance information based on a distance from an imaging section to the object when the imaging section captured the captured image;

excluding a structure that is more global than a desired concavity-convexity part from the distance information based on known characteristic information to extract information about the desired concavity-convexity part as extracted concavity-convexity information, the known characteristic information being information that represents known characteristics relating to a structure of the object, and the extracted concavity-convexity information being concavity-convexity information about the object based on the distance information;

determining whether or not to exclude or reduce the extracted concavity-convexity information corresponding to each given area of the captured image; and

excluding the extracted concavity-convexity information corresponding to the given area for which it has been determined to exclude the extracted concavity-convexity information, or reducing a degree of concavities and convexities represented by the extracted concavity-convexity information corresponding to the given area for which it has been determined to reduce the extracted concavity-convexity information.

According to another aspect of the invention, there is provided a non-transitory information storage device storing an image processing program that causes a computer to perform steps of:

acquiring a captured image that includes an image of an object;

acquiring distance information based on a distance from an imaging section to the object when the imaging section captured the captured image;

excluding a structure that is more global than a desired concavity-convexity part from the distance information based on known characteristic information to extract information about the desired concavity-convexity part as extracted concavity-convexity information, the known characteristic information being information that represents known characteristics relating to a structure of the object, and the extracted concavity-convexity information being concavity-convexity information about the object based on the distance information;

determining whether or not to exclude or reduce the extracted concavity-convexity information corresponding to each given area of the captured image; and

excluding the extracted concavity-convexity information corresponding to the given area for which it has been determined to exclude the extracted concavity-convexity information, or reducing a degree of concavities and convexities represented by the extracted concavity-convexity information corresponding to the given area for which it has been determined to reduce the extracted concavity-convexity information.

### BRIEF DESCRIPTION OF THE DRAWINGS

FIG. 1 illustrates a configuration example of an image processing device.

FIG. 2 illustrates a configuration example of an endoscope apparatus according to the first embodiment.

FIG. 3 illustrates a configuration example of a Bayer color filter array.

FIG. 4 illustrates an example of the spectral sensitivity characteristics of a red color filter, a blue color filter, and a green color filter.

FIG. 5 illustrates a detailed configuration example of an image processing section according to the first embodiment.

FIG. 6 illustrates a detailed configuration example of a concavity-convexity information acquisition section.

FIGS. 7A to 7C are views illustrating a process that extracts extracted concavity-convexity information using a filtering process.

FIG. 8 is a view illustrating coordinates (x, y) of an image, a distance map, and a concavity-convexity map.

FIG. 9 illustrates a detailed configuration example of a determination section according to the first embodiment.

FIG. 10 is a view illustrating a hue value.

FIG. 11 illustrates a detailed configuration example of a bright spot determination section.

FIG. 12 is a view illustrating a bright spot discrimination process.

FIG. 13 illustrates an example of noise characteristics corresponding to a luminance value.

FIG. 14 illustrates a detailed configuration example of a treatment tool determination section.

FIGS. 15A to 15D are views illustrating a concavity-convexity information correction process.

FIG. 16 illustrates an example of a flowchart of image processing (first embodiment).

FIG. 17 illustrates an example of a flowchart of a necessity determination process (first embodiment).

FIG. 18 illustrates a configuration example of an endoscope apparatus according to the second embodiment.

FIG. 19 illustrates an example of a light source spectrum (second embodiment).

FIG. 20 illustrates a detailed configuration example of an image processing section according to the second embodiment.

FIG. 21 illustrates a detailed configuration example of a determination section according to the second embodiment.

FIG. 22 illustrates an example of a flowchart of image processing (second embodiment).

FIG. 23 illustrates an example of a flowchart of a necessity determination process (second embodiment).

### DESCRIPTION OF EXEMPLARY EMBODIMENTS

Exemplary embodiments of the invention are described below. Note that the following exemplary embodiments do not in any way limit the scope of the invention laid out in the claims. Note also that all of the elements described in connection with the following exemplary embodiments should not necessarily be taken as essential elements of the invention.

Although an example in which corrected extracted concavity-convexity information is used for an enhancement process is described below, the corrected extracted concavity-convexity information may be used for various processes other than the enhancement process. Although an example in which concavity-convexity information that is not useful for the enhancement process (e.g., extracted concavity-convexity information about a residue or a bright spot) is corrected is described below, the correction determination conditions may be set corresponding to a process performed in the subsequent stage.

### 1. Method

When examining the digestive tract using an endoscope apparatus, and determining the presence or absence of an early lesion, or determining the range of an early lesion, weight is attached to minute concavities and convexities of the surface of tissue. A process that enhances a specific spatial frequency is normally used for an endoscope apparatus as an image enhancement process. However, it is difficult to enhance minute concavities and convexities of the surface of tissue using the process that enhances a specific spatial frequency.

In Japan, a dye-spraying method (e.g., a method that sprays indigo carmine) has been used to enhance minute concavities and convexities of the surface of tissue. The contrast of minute concavities and convexities is enhanced by spraying a dye. However, it is troublesome for the doctor to spray a dye. Moreover, the burden imposed on the patient increases due to an increase in examination time. It may be impossible to observe the original state of the surface of tissue after spraying a dye, and an increase in cost may occur by utilizing a dye. In foreign countries, the dye-spraying method is not normally employed in order to avoid a complex operation and reduce cost, and observation that utilizes a normal white light source is normally employed. In this case, an early lesion may be missed.

Therefore, it is advantageous for doctors and patients in Japan if it is possible to provide a method that can enhance the contrast of concavities and convexities of the surface of tissue by image processing without spraying a dye. It is also possible to propose a novel diagnostic technique in foreign countries, and contribute toward preventing a situation in which an early lesion may be missed.

JP-A-2003-88498 discloses a method that simulates a state in which a dye has been sprayed by image processing. The method disclosed in JP-A-2003-88498 compares the luminance level of an attention pixel in a locally extracted area with the luminance level of its peripheral pixel, and colors the attention area when the attention area is darker than the peripheral area. However, the method disclosed in JP-A-2003-88498 is based on the assumption that the object is captured more darkly as the distance to the surface of tissue increases since the intensity of reflected light from the surface of tissue decreases. Therefore, information that is irrelevant to minute concavities and convexities of the surface of tissue (e.g., information about an area around a bright spot, a shadow due to a structure, a blood vessel, or a mucous membrane situated around a blood vessel) may be erroneously detected as the concavity-convexity information.

Specifically, the method enhances a concavity-convexity structure of the object has a problem in that a concavity-convexity structure that need not be enhanced, and a concavity-convexity structure that should not be enhanced, are also enhanced.

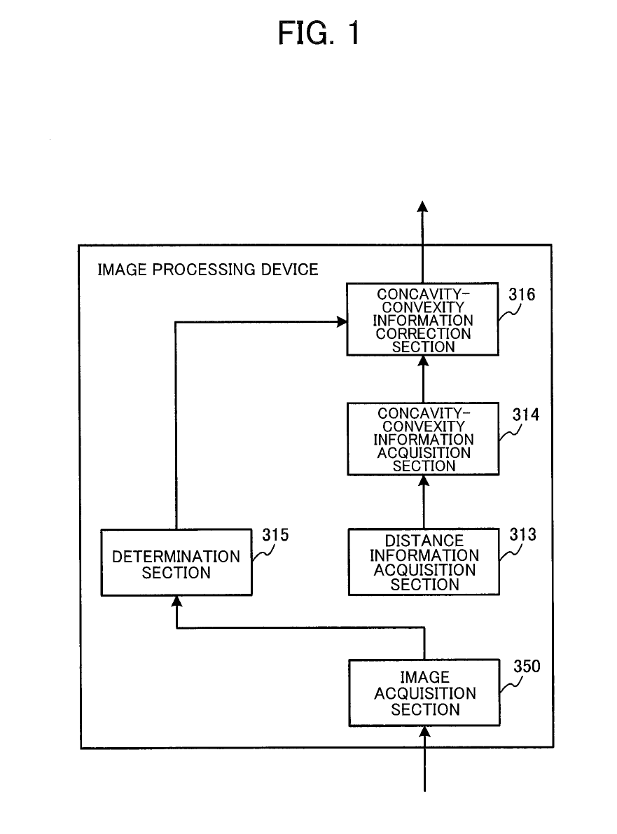

FIG. 1 illustrates a configuration example of an image processing device that can solve the above problem. The image processing device includes an image acquisition section 350 that acquires a captured image that includes an image of an object, a distance information acquisition section 313 that acquires distance information based on the distance from an imaging section to the object when the imaging section captured the captured image, a concavity-convexity information acquisition section 314 that acquires concavity-convexity information about the object based on the distance information as extracted concavity-convexity information, a determination section 315 that determines whether or not to exclude or reduce the extracted concavity-convexity information corresponding to each given area (a pixel or an area having a given size) of the captured image, and a concavity-convexity information correction section 316 that excludes the extracted concavity-convexity information corresponding to the given area for which the determination section 315 has determined to exclude the extracted concavity-convexity information, or reduces the degree of concavities and convexities represented by the extracted concavity-convexity information corresponding to the given area for which the determination section 315 has determined to reduce the extracted concavity-convexity information.

This makes it possible to exclude or reduce the extracted concavity-convexity information about an area that satisfies a given determination condition (e.g., an area that is not required for the process in the subsequent stage, or an area that should not be used for the process in the subsequent stage) from the extracted concavity-convexity information that corresponds to the captured image. When an enhancement process that enhances a concavity-convexity structure of tissue is performed as the process in the subsequent stage, it is possible to perform the enhancement process to the observation target concavity-convexity structure for the user. Specifically, it is possible to suppress a situation in which the enhancement process is performed on a concavity-convexity structure that is not concavities and convexities specific to tissue, or an area other than concavities and convexities is erroneously observed as a concavity-convexity structure due to the enhancement process, for example.

The term “distance information” used herein refers to information in which each position of the captured image is linked to the distance to the object at each position of the captured image. For example, the distance information is a distance map. The term “distance map” used herein refers to a map in which the distance (depth) to the object in the Z-axis direction (i.e., the direction of the optical axis of the imaging section 200 described later) is specified corresponding to each point (e.g., each pixel) in the XY plane, for example.

Note that the distance information may be various types of information that are acquired based on the distance from the imaging section 200 to the object. For example, when implementing triangulation using a stereo optical system, the distance with respect to an arbitrary point of a plane that connects two lenses that produce a parallax may be used as the distance information. When using a Time-of-Flight method, the distance with respect to each pixel position in the plane of the image sensor may be acquired as the distance information, for example. In such a case, the distance measurement reference point is set to the imaging section 200. Note that the distance measurement reference point may be set to an arbitrary position other than the imaging section 200, such as an arbitrary position within the three-dimensional space that includes the imaging section and the object. The distance information acquired using such a reference point is also intended to be included within the term “distance information”.

The distance from the imaging section 200 to the object may be the distance from the imaging section 200 to the object in the depth direction, for example. For example, the distance in the direction of the optical axis of the imaging section 200 may be used. For example, when a viewpoint is set in the direction orthogonal to the optical axis of the imaging section 200, the distance from the imaging section 200 to the object may be the distance observed at the viewpoint (i.e., the distance from the imaging section 200 to the object along a line that passes through the viewpoint and is parallel to the optical axis).

For example, the distance information acquisition section 313 may transform the coordinates of each corresponding point in a first coordinate system in which a first reference point of the imaging section 200 is the origin, into the coordinates of each corresponding point in a second coordinate system in which a second reference point within the three-dimensional space is the origin, using a known coordinate transformation process, and measure the distance based on the coordinates obtained by transformation. In this case, the distance from the second reference point to each corresponding point in the second coordinate system is identical with the distance from the first reference point to each corresponding point in the first coordinate system (i.e., the distance from the imaging section to each corresponding point).

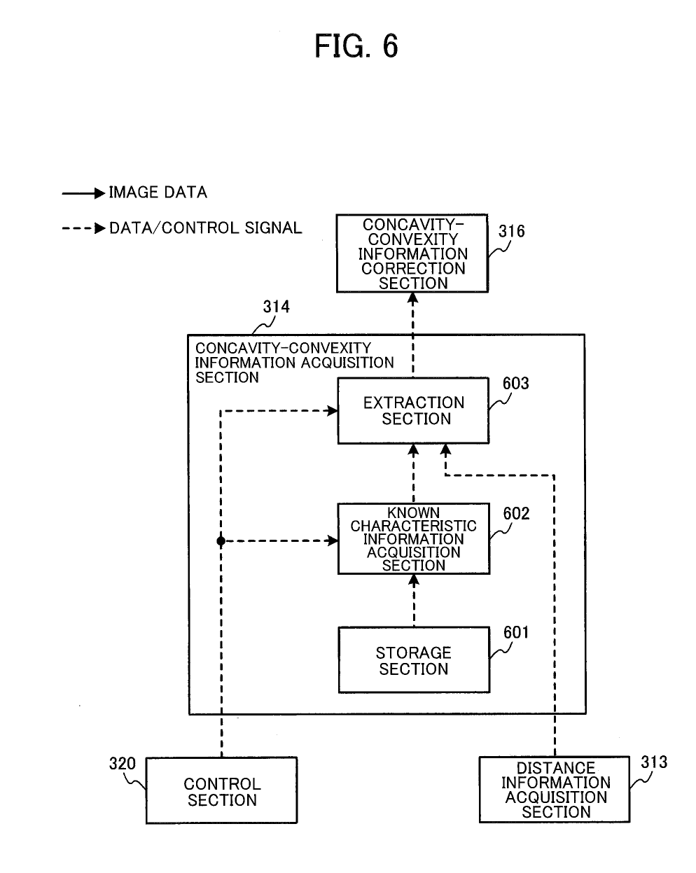

The distance information acquisition section 313 may set a virtual reference point at a position that can maintain a relationship similar to the relationship between the distance values of the pixels on the distance map acquired when setting the reference point to the imaging section 200, to acquire the distance information based on the distance from the imaging section 200 to each corresponding point. For example, when the actual distances from the imaging section 200 to three corresponding points are respectively “3”, “4”, and “5”, the distance information acquisition section 313 may acquire distance information “1.5”, “2”, and “2.5” respectively obtained by halving the actual distances “3”, “4”, and “5” while maintaining the relationship between the distance values of the pixels. When the concavity-convexity information acquisition section 314 acquires the concavity-convexity information using the extraction operation parameter (as described later with reference to FIG. 6 and the like), the concavity-convexity information acquisition section 314 uses a different extraction process parameter as compared with the case where the reference point is set to the imaging section 200. Since it is necessary to use the distance information when determining the extraction process parameter, the extraction process parameter is determined in a different way when the distance measurement reference point has changed (i.e., when the distance information is represented in a different way). For example, when extracting the extracted concavity-convexity information using a morphological process (described later), the size of a structural element (e.g., the diameter of a sphere) used for the extraction process is adjusted, and the concavity-convexity part extraction process is performed using the structural element that has been adjusted in size.

The term “extracted concavity-convexity information” used herein refers to information obtained by extracting information about a specific structure from the distance information. More specifically, the extracted concavity-convexity information refers to information obtained by excluding a global change in distance (i.e., a change in distance due to a lumen structure in a narrow sense) from the distance information.

For example, the concavity-convexity information acquisition section 314 extracts a concavity-convexity part of the object that agrees with characteristics specified by known characteristic information based on the distance information and the known characteristic information that represents known characteristics relating to the structure of the object (e.g., dimensional information that represents the width, the depth, and the like of the concavity-convexity part present on the surface of tissue).

This makes it possible to separate the concavity-convexity information that agrees with the known characteristic information based on the known characteristic information about the desired extraction target concavity-convexity part (e.g., a concavity-convexity part of tissue due to a lesion). Therefore, it is possible to acquire the extracted concavity-convexity information about the desired concavity-convexity part, and use the extracted concavity-convexity information for the process in the subsequent stage (e.g., enhancement process).

Note that the configuration is not limited thereto. It suffices to perform only a process that makes it possible to appropriately perform the process in the subsequent stage (e.g., enhancement process) (i.e., a process that excludes a global structure). Specifically, it is not indispensable to use the known characteristic information when acquiring the extracted concavity-convexity information.

### 2. First Embodiment

### 2.1. Endoscope Apparatus

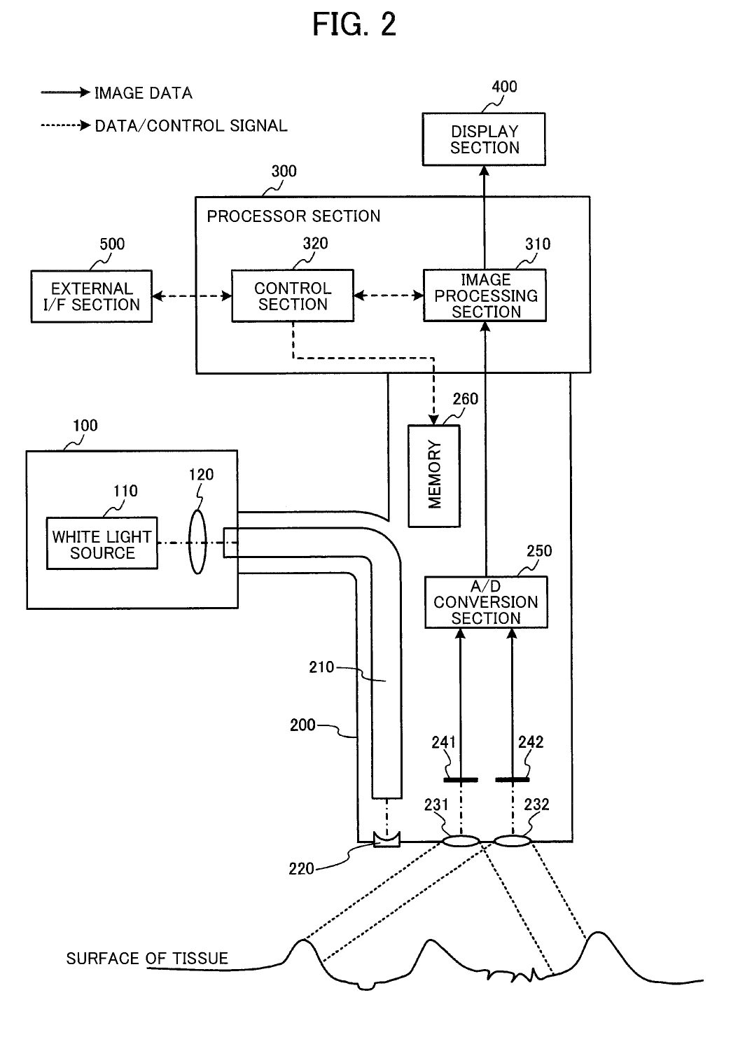

FIG. 2 illustrates a configuration example of an endoscope apparatus according to a first embodiment of the invention. The endoscope apparatus includes a light source section 100, an imaging section 200, a processor section 300 (control device), a display section 400, and an external OF section 500.

The light source section 100 includes a white light source 110, and a condenser lens 120 that focuses white light emitted from the white light source 110 on a light guide fiber 210.

The imaging section 200 is formed to be elongated and flexible so that the imaging section 200 can be inserted into a body cavity, for example. The imaging section 200 includes the light guide fiber 210 that guides the white light emitted from the white light source 110 to the end of the imaging section 200, an illumination lens 220 that diffuses the white light guided by the light guide fiber 210, and applies the diffused white light to the surface of tissue, objective lenses 231 and 232 that focus the light from the surface of tissue, image sensors 241 and 242 that detect the focused light, and an A/D conversion section 250 that converts analog signals photoelectrically converted by the image sensors 241 and 242 into digital signals. The imaging section 200 also includes a memory 260 that stores scope type information (e.g., identification number).

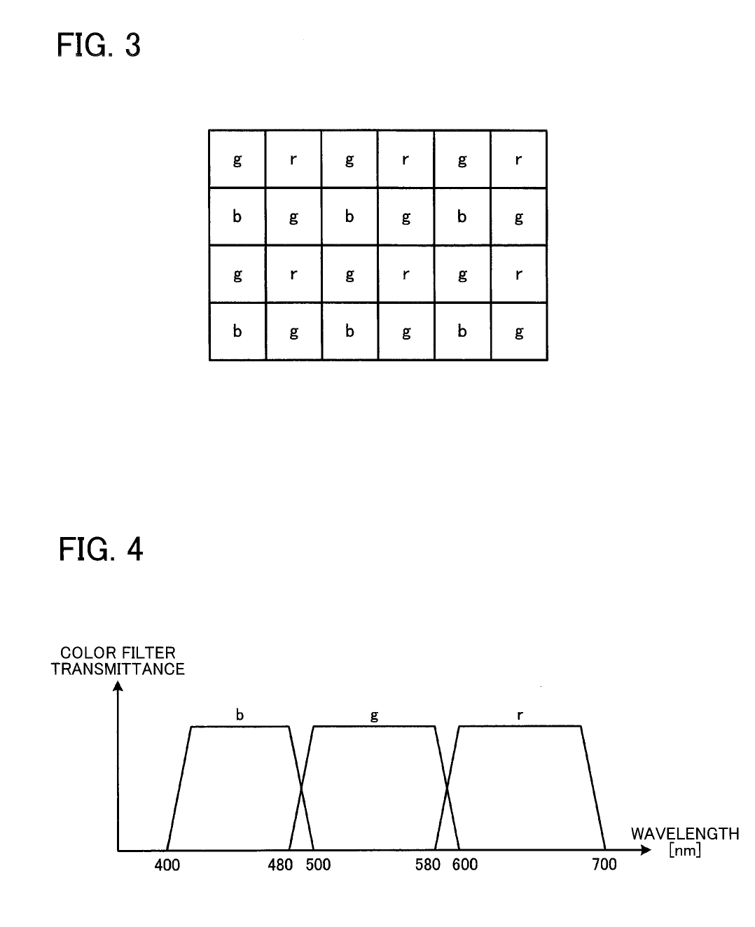

As illustrated in FIG. 3, the image sensors 241 and 242 include a Bayer color filter array, for example. The color filters include a red color filter r, a green color filter g, and a blue color filter b. Each color filter has the spectral sensitivity characteristics illustrated in FIG. 4, for example.

The objective lenses 231 and 232 are disposed at such an interval that a given parallax image (hereinafter referred to as “stereo image”) can be captured. The objective lenses 231 and 232 form an image on the image sensors 241 and 242, respectively. It is possible to acquire distance information about the distance from the end of the imaging section 200 to the surface of tissue by performing a stereo matching process on the stereo image (described later). Note that the image captured by the image sensor 241 is referred to as “left image”, the image captured by the image sensor 242 is referred to as “right image”, and the left image and the right image are collectively referred to as “stereo image”.

The processor section 300 includes an image processing section 310 and a control section 320. The image processing section 310 performs image processing (described later) on the stereo image output from the A/D conversion section 250 to generate a display image, and outputs the display image to the display section 400. The control section 320 controls each section of the endoscope apparatus. For example, the control section 320 controls the operation of the image processing section 310 based on a signal output from the external I/F section 500 (described later).

The display section 400 is a display device that can display the display image output from the processor section 300 as a moving image (movie). The display section 400 is implemented by a cathode-ray tube display (CRT), a liquid crystal monitor, or the like.

The external I/F section 500 is an interface that allows the user to input information and the like to the endoscope apparatus. The external I/F section 500 includes a power switch (power ON/OFF switch), a mode (e.g., imaging mode) switch button, and the like. The external I/F section 500 may include an enhancement process button (not illustrated in the drawings) that allows the user to issue an enhancement process ON/OFF instruction. In this case, the user can issue an enhancement process ON/OFF instruction by operating the enhancement process button. The external IN section 500 outputs an enhancement process ON/OFF instruction signal to the control section 320.

### 2.2. Image Processing Section

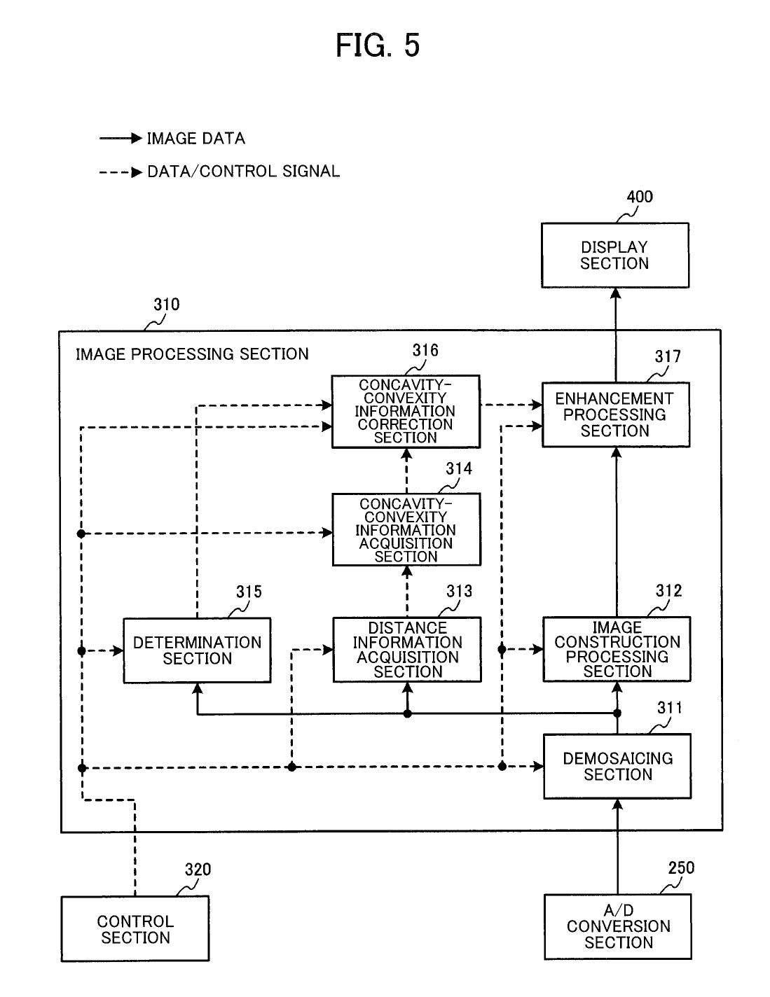

FIG. 5 illustrates a detailed configuration example of the image processing section 310. The image processing device 310 includes a demosaicing section 311, an image construction processing section 312, a distance information acquisition section 313 (distance map acquisition section), a concavity-convexity information acquisition section 314 (concavity-convexity map acquisition section), a determination section 315 (necessity determination section), a concavity-convexity information correction section 316 (concavity-convexity map correction section), and an enhancement processing section 317. The demosaicing section 311 corresponds to the image acquisition section 350 illustrated in FIG. 1.

The A/D conversion section 250 is connected to the demosaicing section 311. The demosaicing section 311 is connected to the image construction processing section 312, the distance information acquisition section 313, and the determination section 315. The distance information acquisition section 313 is connected to the concavity-convexity information acquisition section 314. The determination section 315 and the concavity-convexity information acquisition section 314 are connected to the concavity-convexity information correction section 316. The concavity-convexity information correction section 316 and the image construction processing section 312 are connected to the enhancement processing section 317. The enhancement processing section 317 is connected to the display section 400. The control section 320 is connected to the demosaicing section 311, the image construction processing section 312, the distance information acquisition section 313, the concavity-convexity information acquisition section 314, the determination section 315, the concavity-convexity information correction section 316, and the enhancement processing section 317, and controls the demosaicing section 311, the image construction processing section 312, the distance information acquisition section 313, the concavity-convexity information acquisition section 314, the determination section 315, the concavity-convexity information correction section 316, and the enhancement processing section 317.

The demosaicing section 311 performs a demosaicing process on the stereo image output from the A/D conversion section 250. Since the image sensors 241 and 242 include the Bayer color filter array, each pixel has only an R, G, or B signal. Therefore, an RGB image is generated using a known bicubic interpolation process or the like. The demosaicing section 311 outputs the stereo image subjected to the demosaicing process to the image construction processing section 312, the distance information acquisition section 313, and the determination section 315.

The image construction processing section 312 performs a known WB process, a known γ process, and the like on the stereo image output from the demosaicing section 311, and outputs the resulting stereo image to the enhancement processing section 317.

The distance information acquisition section 313 performs the stereo matching process on the stereo image output from the demosaicing section 311 to acquire the distance information about the distance from the end of the imaging section 200 to the surface of tissue. Specifically, the distance information acquisition section 313 performs a block matching process on the left image (reference image) and the right image with respect to the processing target pixel and its peripheral area (i.e., a block having a given size) using an epipolar line that passes through the processing target pixel of the reference image. The distance information acquisition section 313 detects a position at which the correlation obtained by the block matching process becomes a maximum as a parallax, and converts the parallax into the distance in the depth direction. This conversion process includes a process that corrects the optical magnification of the imaging section 200. For example, the distance information acquisition section 313 sequentially shifts the processing target pixel by one pixel, and acquires a distance map having the same number of pixels as that of the stereo image as the distance information. The distance information acquisition section 313 outputs the distance map to the concavity-convexity information acquisition section 314. Note that the right image may be used as the reference image.

The concavity-convexity information extraction section 314 extracts concavity-convexity information that represents the concavity-convexity parts of the surface of tissue (excluding the distance information that depends on the shape of the digestive tract (e.g., lumen and folds)) from the distance information, and outputs the concavity-convexity information to the concavity-convexity information correction section 316 as extracted concavity-convexity information. Specifically, the concavity-convexity information acquisition section 314 extracts a concavity-convexity part that has the desired dimensional characteristics based on known characteristic information that represents the size (i.e., dimensional information such as width, height, or depth) of the extraction target concavity-convexity part. The details of the concavity-convexity information acquisition section 314 are described later.

The determination section 315 determines an area for which the extracted concavity-convexity information is excluded or reduced based on whether or not the feature quantity (e.g., hue value or edge quantity) of the image satisfies a given condition. Specifically, the determination section 315 detects a pixel that corresponds to a residue, a treatment tool, or the like as a pixel for which it is unnecessary to acquire the extracted concavity-convexity information. The determination section 315 detects a pixel that corresponds to a flat area, a dark area, a bright spot, or the like as a pixel for which it is difficult to generate the distance map (i.e., the reliability of the distance map is low). The determination section 315 outputs position information about the detected pixel to the concavity-convexity information correction section 316. The details of the determination section 315 are described later. Note that the determination process may be performed on a pixel basis, or the captured image may be divided into a plurality of blocks having a given size, and the determination process may be performed on a block basis.

The concavity-convexity information correction section 316 excludes the extracted concavity-convexity information, or reduces the degree of concavity and convexity corresponding to an area for which it has been determined to exclude or reduce the extracted concavity-convexity information (hereinafter referred to as “exclusion target area”). For example, since the extracted concavity-convexity information about a flat area represents a constant value (constant distance), the extracted concavity-convexity information about the exclusion target area is excluded by setting the extracted concavity-convexity information to the constant value. Alternatively, the degree of concavity and convexity in the exclusion target area is reduced by performing a smoothing filtering process on the extracted concavity-convexity information about the exclusion target area. The details of the concavity-convexity information correction section 316 are described later.

The enhancement processing section 317 performs an enhancement process on the captured image based on the extracted concavity-convexity information, and outputs the resulting image to the display section 400 as a display image. For example, the enhancement processing section 317 performs a process that increases the degree of blueness on an area of the captured image that corresponds to a recess formed in tissue. This makes it possible to enhance the concavities and convexities of the surface area of tissue without spraying a dye.

### 2.3. Concavity-Convexity Information Acquisition Process

FIG. 6 illustrates a detailed configuration example of the concavity-convexity information acquisition section 314. The concavity-convexity information acquisition section 314 includes a storage section 601, a known characteristic information acquisition section 602, and an extraction section 603. An example in which the frequency characteristics of a low-pass filtering process are set based on the known characteristic information that represents the size of a concavity-convexity part is described below. Note that the configuration is not limited thereto. For example, predetermined frequency characteristics may be set to the low-pass filter. In this case, the storage section 601 and the known characteristic information acquisition section 602 may be omitted.

The known characteristic information acquisition section 602 acquires the dimensional information (i.e., information about the size of the extraction target concavity-convexity part of tissue) from the storage section 601 as the known characteristic information, and determines the frequency characteristics of the low-pass filtering process based on the dimensional information. The extraction section 603 performs the low-pass filtering process on the distance map using the determined frequency characteristics to extract shape information about a lumen, folds, and the like. The extraction section 603 subtracts the shape information from the distance map to generate a concavity-convexity map of the surface area of tissue (i.e., information about a concavity-convexity part having the desired size), and outputs the concavity-convexity map to the concavity-convexity information correction section 316 as the extracted concavity-convexity information.

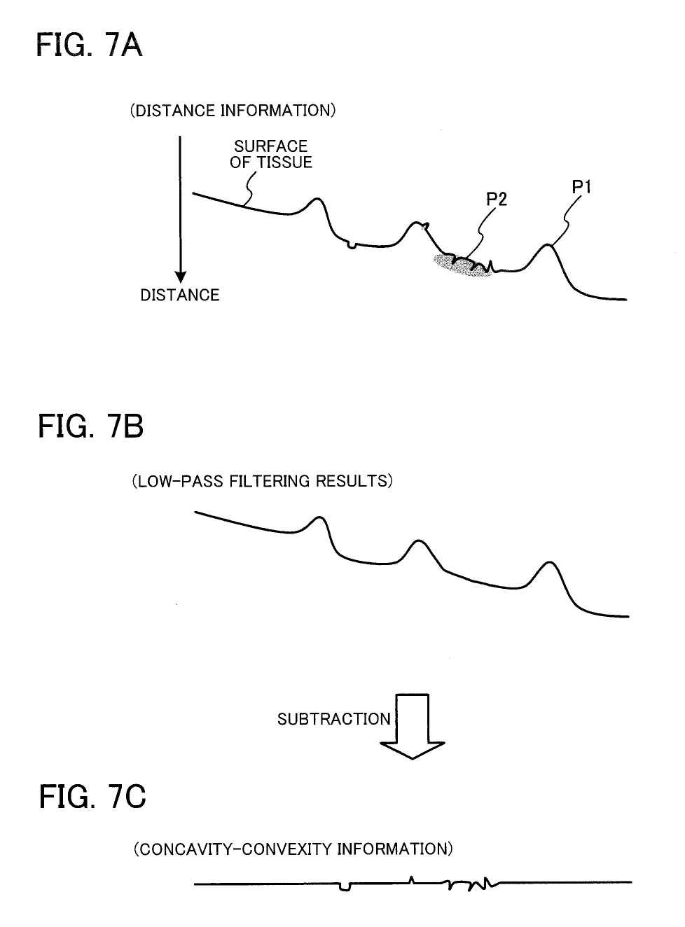

FIG. 7A schematically illustrates an example of the distance map. FIG. 7A illustrates an example in which the distance map is a one-dimensional distance map for convenience of explanation. In FIG. 7A, the arrow indicates the distance axis. The distance map includes information about a rough structure of tissue (e.g., the shape information about a lumen and folds) (see P1), and information about concavity-convexity parts of the surface area of tissue (see P2). As illustrated in FIG. 7B, the extraction section 603 performs the low-pass filtering process on the distance map to extract the information about the rough structure of tissue. As illustrated in FIG. 7C, the extraction section 603 subtracts the information about the rough structure of tissue from the distance map to generate the concavity-convexity map that is the concavity-convexity information about the surface area of tissue.

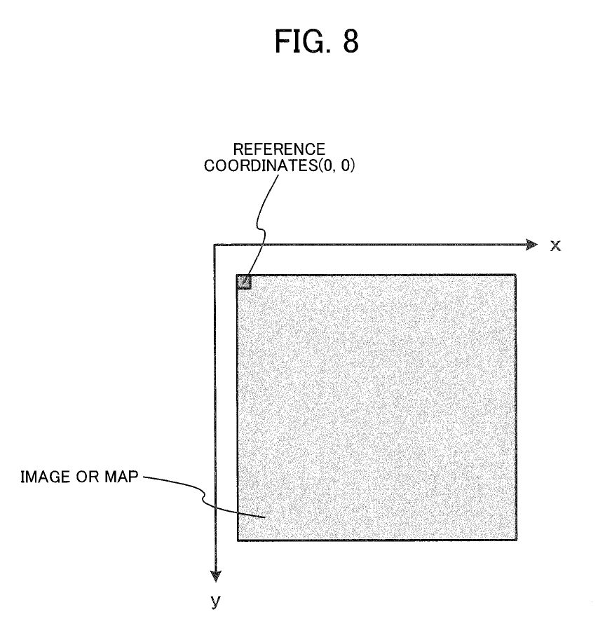

As illustrated in FIG. 8, the horizontal direction of the image, the distance map, and the concavity-convexity map is defined as an x-axis, and the vertical direction of the image, the distance map, and the concavity-convexity map is defined as a y-axis. The upper left corner of the image (or the map) is defined as reference coordinates (0, 0). The distance at the coordinates (x, y) of the distance map is defined as dist(x, y), and the distance (shape information) at the coordinates (x, y) of the distance map after the low-pass filtering process is defined as dist_LPF(x, y). In this case, the concavity-convexity information diff(x, y) at the coordinates (x, y) of the concavity-convexity map is calculated by the following expression (1).

diff(x,y)=dist(x,y)−dist_{—}LP_{—}F(x,y) (1)

A process that determines the cut-off frequency (extraction process parameter in a broad sense) from the dimensional information is described in detail below.

The known characteristic information acquisition section 602 acquires the size (i.e., dimensional information (e.g., width, height, or depth)) of the extraction target concavity-convexity part of tissue due to a lesion, the size (i.e., dimensional information (e.g., width, height, or depth)) of the lumen and the folds specific to the observation target part based on observation target part information, and the like from the storage section 601.

Note that the observation target part information is information that represents the observation target part that is determined based on scope ID information, for example. The observation target part information may also be included in the known characteristic information. For example, when the scope is an upper gastrointestinal scope, the observation target part is the gullet, the stomach, or the duodenum. When the scope is a lower gastrointestinal scope, the observation target part is the large intestine. Since the dimensional information about the extraction target concavity-convexity part and the dimensional information about the lumen and the folds specific to the observation target part differ corresponding to each part, the known characteristic information acquisition section 602 outputs information about the typical size of a lumen and folds acquired based on the observation target part information to the extraction section 603, for example.

The extraction section 603 performs the low-pass filtering process using a given size (e.g., N×N pixels (N is a natural number equal to or larger than 2)) on the input distance information. The extraction section 603 adaptively determines the extraction process parameter based on the resulting distance information (local average distance). Specifically, the extraction section 603 determines the characteristics of the low-pass filtering process that smooth the extraction target concavity-convexity part of tissue due to a lesion while maintaining the structure of the lumen and the folds specific to the observation target part. Since the characteristics of the extraction target (i.e., concavity-convexity part) and the exclusion target (i.e., folds and lumen) can be determined from the known characteristic information, the spatial frequency characteristics are known, and the characteristics of the low-pass filter can be determined. Since the apparent size of the structure changes corresponding to the local average distance, the characteristics of the low-pass filter are determined corresponding to the local average distance.

The low-pass filtering process is implemented by a Gaussian filter represented by the following expression (2), or a bilateral filter represented by the following expression (3), for example. Note that p(x) is the distance at the coordinate x on the distance map. Although the expressions (2) and (3) represent a one-dimensional filter for convenience of explanation, a two-dimensional filter (coordinates (x, y)) is applied in actual applications. The frequency characteristics of these filters are controlled using σ, σ_{c}, and σᵥ. A σ map that corresponds to the pixels of the distance map on a one-to-one basis may be generated as the extraction process parameter. When using the bilateral filter, a σ_{c} map and/or a σᵥ map may be generated as the extraction process parameter.

$$\begin{matrix}f ( x ) = \frac{1}{N} exp ( - \frac{{( x - x 0 )}^{2}}{2 {σ}^{2}} ) & ( 2 ) \\ f ( x ) = \frac{1}{N} exp ( - \frac{{( x - x 0 )}^{2}}{2 {σ}_{c}^{2}} ) \times exp ( - \frac{{( p ( x ) - p ( x 0 ) )}^{2}}{2 {σ}_{v}^{2}} ) & ( 3 )\end{matrix}$$

For example, a may be a value that is larger than a value obtained by multiplying the pixel-to-pixel distance D1 of the distance map corresponding to the size of the extraction target concavity-convexity part by α (>1), and is smaller than a value obtained by multiplying the pixel-to-pixel distance D2 of the distance map corresponding to the size of the lumen and the folds specific to the observation target part by β (<1). For example, a may be calculated by σ=(α*D1+β*D2)/2*Rσ. Note that Rσ is a function of the local average distance. The value Rσ increases as the local average distance decreases, and decreases as the local average distance increases.

Note that the first embodiment is not limited to the extraction process that utilizes the low-pass filtering process. For example, the extracted concavity-convexity information may be acquired using a morphological process. When using the morphological process, an opening process and a closing process using a given kernel size (i.e., the size (sphere diameter) of the structural element) are performed on the distance map. The extraction process parameter is the size of the structural element. For example, when using a sphere as the structural element, the diameter of the sphere is set to be smaller than the size of the lumen and the folds specific to the observation target part based on the observation target part information, and larger than the size of the extraction target concavity-convexity part of tissue due to a lesion. The diameter of the sphere is increased as the local average distance decreases, and is decreased as the local average distance increases. The recesses formed in the surface of tissue are extracted by calculating the difference between information obtained by the closing process and the original distance information. The protrusions formed on the surface of tissue are extracted by calculating the difference between information obtained by the opening process and the original distance information.

According to the first embodiment, the concavity-convexity information acquisition section 314 determines the extraction process parameter based on the known characteristic information, and extracts a concavity-convexity part of the object as the extracted concavity-convexity information based on the determined extraction process parameter.

This makes it possible to perform the extracted concavity-convexity information extraction process (e.g., separation process) using the extraction process parameter determined based on the known characteristic information. The extraction process may be performed using the morphological process, the filtering process, or the like. In order to accurately extract the extracted concavity-convexity information, it is necessary to perform a control process that extracts information about the desired concavity-convexity part from the information about various structures included in the distance information while excluding other structures (e.g., the structures specific to tissue, such as folds). In the first embodiment, such a control process is implemented by setting the extraction process parameter based on the known characteristic information.

The captured image may be an in vivo image that is obtained by capturing the inside of a living body, and the known characteristic information acquisition section 602 may acquire part information and concavity-convexity characteristic information as the known characteristic information, the part information being information that represents a part of the living body to which the object corresponds, and the concavity-convexity characteristic information being information about a concavity-convexity part of the living body. The concavity-convexity information acquisition section 314 may determine the extraction operation parameter based on the part information and the concavity-convexity characteristic information.

This makes it possible to acquire the part information about a part (object) within an in vivo image as the known characteristic information when applying the method according to the first embodiment to an in vivo image (e.g., when applying the image processing device according to the first embodiment to a medical endoscope apparatus). When applying the method according to the first embodiment to an in vivo image, it is considered that a concavity-convexity structure that is useful for detecting an early lesion or the like is extracted as the extracted concavity-convexity information. However, the characteristics (e.g., dimensional information) of a concavity-convexity part specific to an early lesion may differ corresponding to each part. Moreover, the exclusion target structure (e.g., folds) necessarily differs corresponding to each part. Therefore, it is necessary to perform an appropriate process corresponding to each part when applying the method according to the first embodiment to an in vivo image. In the first embodiment, such a process is performed based on the part information.

The concavity-convexity information acquisition section 314 may determine the size of the structural element used for the opening process and the closing process as the extraction process parameter based on the known characteristic information, and perform the opening process and the closing process using the structural element having the determined size to extract a concavity-convexity part of the object as the extracted concavity-convexity information.

This makes it possible to extract the extracted concavity-convexity information based on the opening process and the closing process (morphological process in a broad sense). In this case, the extraction process parameter is the size of the structural element used for the opening process and the closing process. When the structural element is a sphere, the extraction process parameter is a parameter that represents the diameter of the sphere, for example.

### 2.4. Necessity Determination Process

If a determination as to whether or not to exclude or reduce the concavity-convexity map (extracted concavity-convexity information) (hereinafter referred to as “necessity determination”) is not performed, the concavity-convexity map of an area (e.g., residue or treatment tool) that is irrelevant to a diagnosis is also generated, and a recess due to a residue or a treatment tool is also enhanced in blue by the enhancement processing section 317. In this case, an image that is very difficult to observe is generated.

When acquiring the distance map (distance information) using the stereo matching process, it is difficult to generate the distance map in a stable manner corresponding to a flat area (in which a structure is not present on the surface of tissue), or a dark area that includes a large amount of noise. Since a bright spot is a specular reflection component of light emitted from the light source, and differs between the left image and the right image, it is difficult to acquire an accurate distance using the stereo matching process. Therefore, concavities and convexities that are not present may be acquired as the concavity-convexity map corresponding to these areas. When such erroneous detection has occurred, a flat area may be enhanced in blue as if concavities and convexities were present, and lead to a misdiagnosis.

As described above, an image that is difficult for the doctor to observe may be generated, and lead to misdiagnosis if the concavity-convexity map is used directly for the enhancement process.

In the first embodiment, the determination section 315 performs a concavity-convexity map necessity determination process. Specifically, the determination section 315 determines a pixel that corresponds to a residue, a treatment tool, or the like to be a pixel for which it is unnecessary to acquire the concavity-convexity map, and determines a pixel that corresponds to a flat area, a dark area, a bright spot, or the like to be a pixel for which it is difficult to generate the distance map. The concavity-convexity information correction section 316 performs a process that excludes or reduces the concavity-convexity information included in the concavity-convexity map corresponding to these pixels.

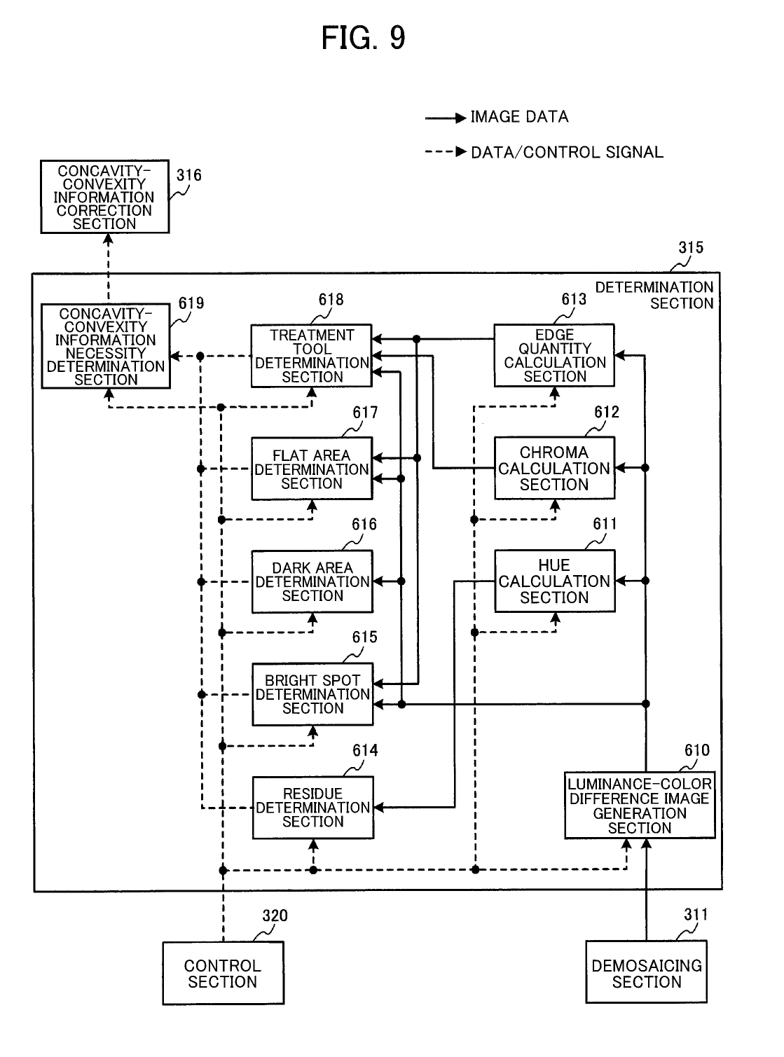

FIG. 9 illustrates a detailed configuration example of the determination section 315. The determination section 315 includes a luminance-color difference image generation section 610 (luminance calculation section), a hue calculation section 611, a chroma calculation section 612, an edge quantity calculation section 613, a residue determination section 614, a bright spot determination section 615, a dark area determination section 616, a flat area determination section 617, a treatment tool determination section 618, and a concavity-convexity information necessity determination section 619.

The demosaicing section 311 is connected to the luminance-color difference image generation section 610. The luminance-color difference image generation section 610 is connected to the hue calculation section 611, the chroma calculation section 612, the edge quantity calculation section 613, the bright spot determination section 615, the dark area determination section 616, the flat area determination section 617, and the treatment tool determination section 618. The hue calculation section 611 is connected to the residue determination section 614. The chroma calculation section 612 is connected to the treatment tool determination section 618. The edge quantity calculation section 613 is connected to the bright spot determination section 615, the flat area determination section 617, and the treatment tool determination section 618. The residue determination section 614, the bright spot determination section 615, the dark area determination section 616, the flat area determination section 617, and the treatment tool determination section 618 are respectively connected to the concavity-convexity information necessity determination section 619. The concavity-convexity information necessity determination section 619 is connected to the concavity-convexity information correction section 316. The control section 320 is connected to the luminance-color difference image generation section 610, the hue calculation section 611, the chroma calculation section 612, the edge quantity calculation section 613, the residue determination section 614, the bright spot determination section 615, the dark area determination section 616, the flat area determination section 617, the treatment tool determination section 618, and the concavity-convexity information necessity determination section 619, and controls the luminance-color difference image generation section 610, the hue calculation section 611, the chroma calculation section 612, the edge quantity calculation section 613, the residue determination section 614, the bright spot determination section 615, the dark area determination section 616, the flat area determination section 617, the treatment tool determination section 618, and the concavity-convexity information necessity determination section 619.

The luminance-color difference image generation section 610 calculates a YCbCr image (luminance-color difference image) based on the RGB image (reference image) output from the demosaicing section 311, and outputs the YCbCr image to the hue calculation section 611, the chroma calculation section 612, the edge quantity calculation section 613, the bright spot determination section 615, and the dark area determination section 616. The YCbCr image is calculated using the following expression (4).

Y(x,y)=0.213×R(x,y)+0.715×G(x,y)+0.072×B(x,y)

Cb(x,y)=−0.114×R(x,y)−0.386×G(x,y)+0.500×B(x,y)

Cr(x,y)=0.500×R(x,y)−0.454×G(x,y)−0.046×B(x,y) (4)

Note that R(x, y), G(x, y), and B(x, y) are respectively the R signal value, the G signal value, and the B signal value at the coordinates (x, y). Y(x, y), Cb(x, y), and Cr(x, y) are respectively the Y signal value, the Cb signal value, and the Cr signal value at the coordinates (x, y).

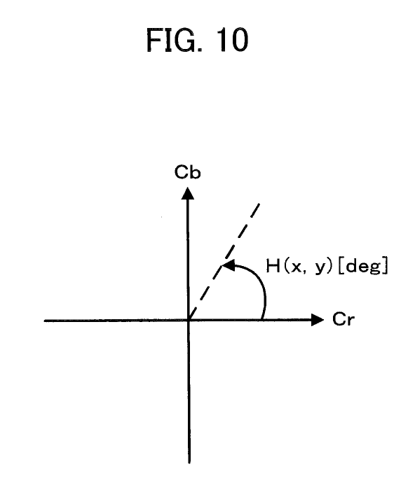

The hue calculation section 611 calculates the hue value H(x, y) (deg) of each pixel of the YCbCr image, and outputs the hue value H(x, y) to the residue determination section 614. As illustrated in FIG. 10, the hue value H(x, y) is defined by an angle (0 to 359) in the CrCb plane. The hue value H(x, y) is calculated using the following expressions (5) to (11).

Specifically, when Cr=0, the hue value H(x, y) is calculated using the expressions (5) to (7). When Cb=0, the hue value H(x, y) is calculated using the expression (5). When Cb>0, the hue value H(x, y) is calculated using the expression (6). When Cb<0, the hue value H(x, y) is calculated using the expression (7).

H(x,y)=0 (5)

H(x,y)=90 (6)

H(x,y)=270 (7)

When Cr≠1, the hue value H(x, y) is calculated using the expressions (8) to (11). Note that “tan⁻¹( )” in the expressions (8) to (11) is a function that returns the inverse arc tangent (deg) of the value in parentheses. “|V|” is a process that acquires the absolute value of a real number V. The expression (8) is used when Cr>0 and Cb>0 (first quadrant), the expression (9) is used when Cr<0 and Cb>0 (second quadrant), the expression (10) is used when Cr<0 and Cb<0 (third quadrant), and the expression (11) is used when Cr>0 and Cb<0 (fourth quadrant).

$$\begin{matrix}H ( x , y ) = {tan}^{- 1} \{ \frac{Cb ( x , y )}{Cr ( x , y )} \} & ( 8 ) \\ H ( x , y ) = 180 - {tan}^{- 1} \{ \frac{Cb ( x , y )}{\vert Cr ( x , y ) \vert} \} & ( 9 ) \\ H ( x , y ) = 180 + {tan}^{- 1} \{ \frac{\vert Cb ( x , y ) \vert}{\vert Cr ( x , y ) \vert} \} & ( 10 ) \\ H ( x , y ) = 360 - {tan}^{- 1} \{ \frac{\vert Cb ( x , y ) \vert}{Cr ( x , y )} \} & ( 11 )\end{matrix}$$

When H(x, y)=360 (deg), the hue value H(x, y) is set to 0 (deg).

The chroma calculation section 612 calculates the chroma value S(x, y) of each pixel of the YCbCr image, and outputs the chroma value S(x, y) to the treatment tool determination section 618. The chroma value S(x, y) is calculated using the following expression (12), for example.

S(x,y)=\sqrt{Cb(x,y)²+Cr(x,y)²} (12)

The edge quantity calculation section 613 calculates the edge quantity E(x, y) of each pixel of the YCbCr image, and outputs the edge quantity E(x, y) to the bright spot determination section 615, the flat area determination section 617, and the treatment tool determination section 618. The edge quantity is calculated using the following expression (13), for example.

$$E ( x , y ) = \vert {\sum }_{i = - 1}^{1} {\sum }_{j = - 1}^{1} {C}_{i , j} \times Y ( x + i , y + j ) \vert , \\ {C}_{i , j} = \{ \begin{matrix}8 & ( i = j = 0 ) \\ - 1 & ( else )\end{matrix} \qquad ( 13 )$$

The residue determination section 614 determines a pixel of the reference image that corresponds to a residue based on the hue value H(x, y) calculated by the hue calculation section 611, and outputs the determination results to the concavity-convexity information necessity determination section 619 using a determination signal. The determination signal may be set to “0” or “1”, for example. Specifically, the determination signal is set to “1” corresponding to a pixel that has been determined to correspond to a residue, and set to “0” corresponding to a pixel other than the pixel that has been determined to correspond to a residue.

While tissue normally has a red color (hue value: 0 to 20 and 340 to 359 (deg)), a residue has a yellow color (hue value: 270 to 310 (deg)). Therefore, a pixel having a hue value H(x, y) of 270 to 310 (deg) is determined to be a residue, for example.

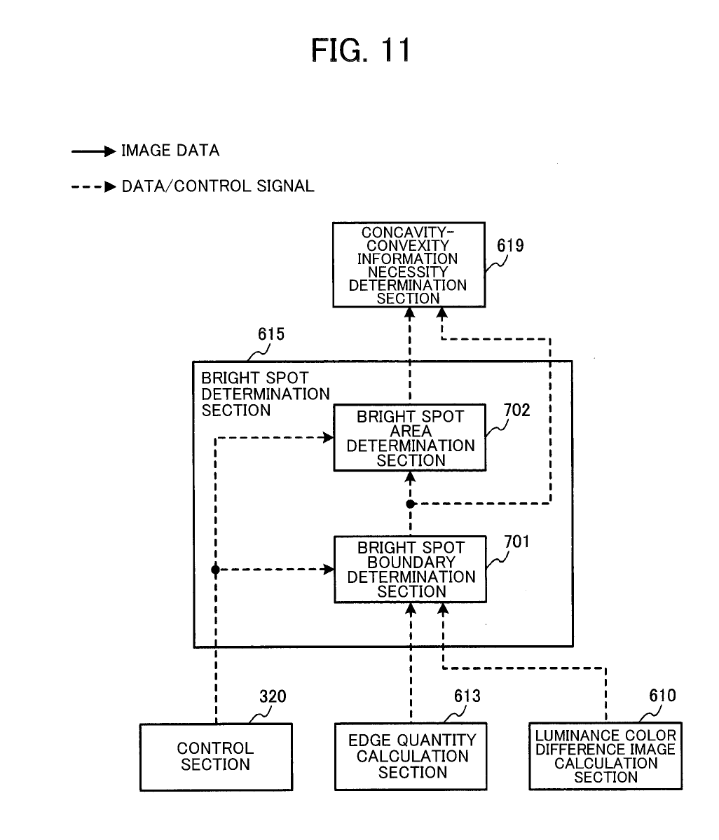

FIG. 11 illustrates a detailed configuration example of the bright spot determination section 615. The bright spot determination section 615 includes a bright spot boundary determination section 701 and a bright spot area determination section 702.

The luminance-color difference image generation section 610 and the edge quantity calculation section 613 are connected to the bright spot boundary determination section 701. The bright spot boundary determination section 701 is connected to the bright spot area determination section 702. The bright spot boundary determination section 701 and the bright spot area determination section 702 are connected to the concavity-convexity information necessity determination section 619. The control section 320 is connected to the bright spot boundary determination section 701 and the bright spot area determination section 702, and controls the bright spot boundary determination section 701 and the bright spot area determination section 702.

The bright spot boundary determination section 701 determines a pixel of the reference image that corresponds to a bright spot based on the luminance value Y(x, y) output from the luminance-color difference image generation section 610 and the edge quantity E(x, y) output from the edge quantity calculation section 613, and outputs the determination results to the concavity-convexity information necessity determination section 619 using a determination signal. The determination signal is set to “1” corresponding to a pixel that has been determined to correspond to a bright spot, and set to “0” corresponding to a pixel other than the pixel that has been determined to correspond to a bright spot, for example. The bright spot boundary determination section 701 outputs the coordinates (x, y) of each pixel that has been determined to correspond to a bright spot to the bright spot area determination section 702 and the concavity-convexity information necessity determination section 619.

The bright spot determination method is described in detail below. A bright spot has a large luminance value Y(x, y) and a large edge quantity E(x, y). Therefore, a pixel for which the luminance value Y(x, y) is larger than a given threshold value th_Y and the edge quantity E(x, y) is larger than a given threshold value th_E1, is determined to correspond to a bright spot. Specifically, a pixel that satisfies the expression (14) is determined to correspond to a bright spot.

Y(x,y)>th_{—}Y and E(x,y)>th_{—}E1 (14)

Specifically, a large edge quantity E(x, y) is observed only at the (bright spot boundary) between a bright spot and tissue, and the edge quantity E(x, y) is small in the inner area (bright spot center area) of a bright spot enclosed by the bright spot boundary. Therefore, if a bright spot is determined based only on the luminance value Y(x, y) and the edge quantity E(x, y), only pixels that correspond to the bright spot boundary are determined to be a bright spot, and pixels that correspond to the bright spot center area are not determined to be a bright spot. According to the first embodiment, the bright spot area determination section 702 determines pixels that correspond to the bright spot center area to be a bright spot.

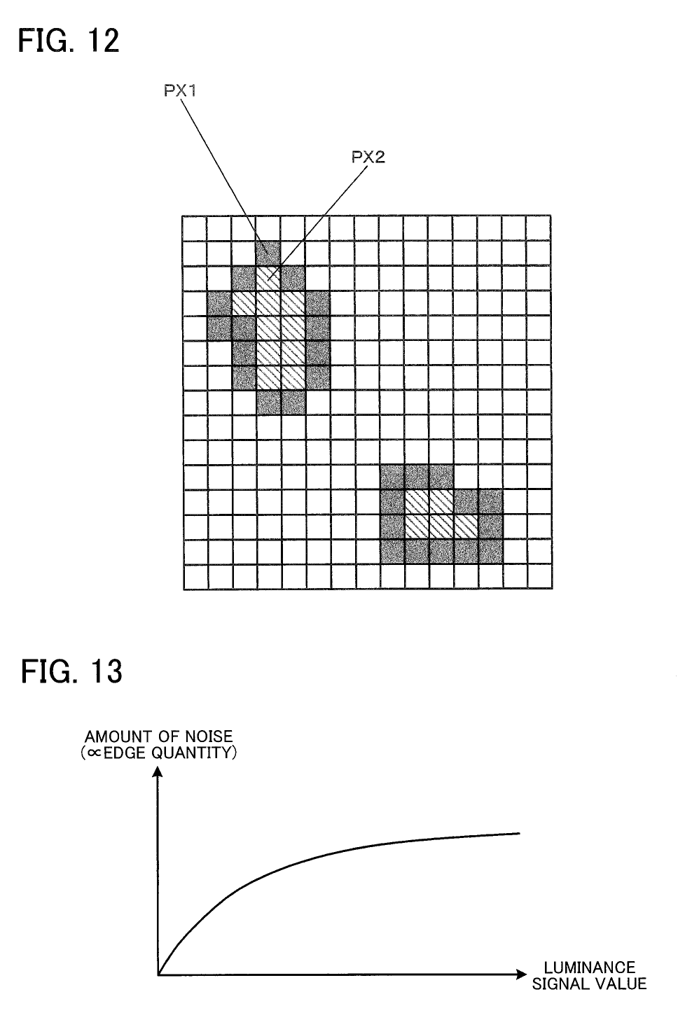

As illustrated in FIG. 12, the bright spot boundary determination section 701 determines pixels PX1 (hatched in gray) that correspond to the bright spot boundary to be a bright spot. The bright spot area determination section 702 determines pixels PX2 (indicated by slanted lines) enclosed by the pixels PX1 to be a bright spot, and outputs the determination results to the concavity-convexity information necessity determination section 619 using a determination signal. The determination signal is set to “1” corresponding to a pixel that has been determined to correspond to a bright spot, and set to “0” corresponding to a pixel other than the pixel that has been determined to correspond to a bright spot, for example.

The dark area determination section 616 determines a pixel of the reference image that corresponds to a dark area based on the luminance value Y(x, y), and outputs the determination results to the concavity-convexity information necessity determination section 619 using a determination signal. The determination signal is set to “1” corresponding to a pixel that has been determined to correspond to a dark area, and set to “0” corresponding to a pixel other than the pixel that has been determined to correspond to a dark area, for example. Specifically, the dark area determination section 616 determines a pixel for which the luminance value Y(x, y) is smaller than a given threshold value th_dark to be a dark area (see the following expression (15)).

Y(x,y)<th_dark (15)

The flat area determination section 617 determines a pixel of the reference image that corresponds to a flat area based on the edge quantity E(x, y), and outputs the determination results to the concavity-convexity information necessity determination section 619 using a determination signal. The determination signal is set to “1” corresponding to a pixel that has been determined to correspond to a flat area, and set to “0” corresponding to a pixel other than the pixel that has been determined to correspond to a flat area, for example. Specifically, the flat area determination section 617 determines a pixel for which the edge quantity E(x, y) is smaller than a given threshold value th_E2(x, y) to be a flat area (see the following expression (16)).

E(x,y)<th_{—}E2(x,y) (16)

The edge quantity E(x, y) in a flat area depends on the amount of noise included in the image. The amount of noise is defined as the standard deviation of the luminance value within a given area. Since the amount of noise normally increases as the brightness (luminance value) of the image increases, it is difficult to determine a flat area using a fixed threshold value. According to the first embodiment, the threshold value th_E2(x, y) is adaptively set corresponding to the luminance value Y(x, y).

Specifically, the edge quantity E(x, y) in a flat area increases in proportion to the amount of noise included in the image. The amount of noise depends on the luminance value Y(x, y), and normally has the characteristics illustrated in FIG. 13. Therefore, the flat area determination section 617 stores the characteristics of the luminance value and the amount of noise illustrated in FIG. 13 as prior information (noise model), and sets the threshold value th_E2(x, y) using the following expression (17) based on the noise model and the luminance value Y(x, y).

th_{—}E2(x,y)=co_{—}NE×noise{Y(x,y)} (17)

Note that noise {Y(x, y)} is a function that returns the amount of noise corresponding to the luminance value Y(x, y) (i.e., the characteristics illustrated in FIG. 13). co_NE is a coefficient for converting the amount of noise into the edge quantity.

The noise model has different characteristics corresponding to the type of the imaging section (scope). For example, the control section 320 may determine the type of the connected scope by referring to the identification number stored in the memory 260 included in the imaging section 200. The flat area determination section 617 may select the noise model based on a signal (type of scope) transmitted from the control section 320.

Although an example in which the amount of noise is calculated based on the luminance value of each pixel has been described above, the configuration is not limited thereto. For example, the amount of noise may be calculated based on the average value of the luminance values within a given area.

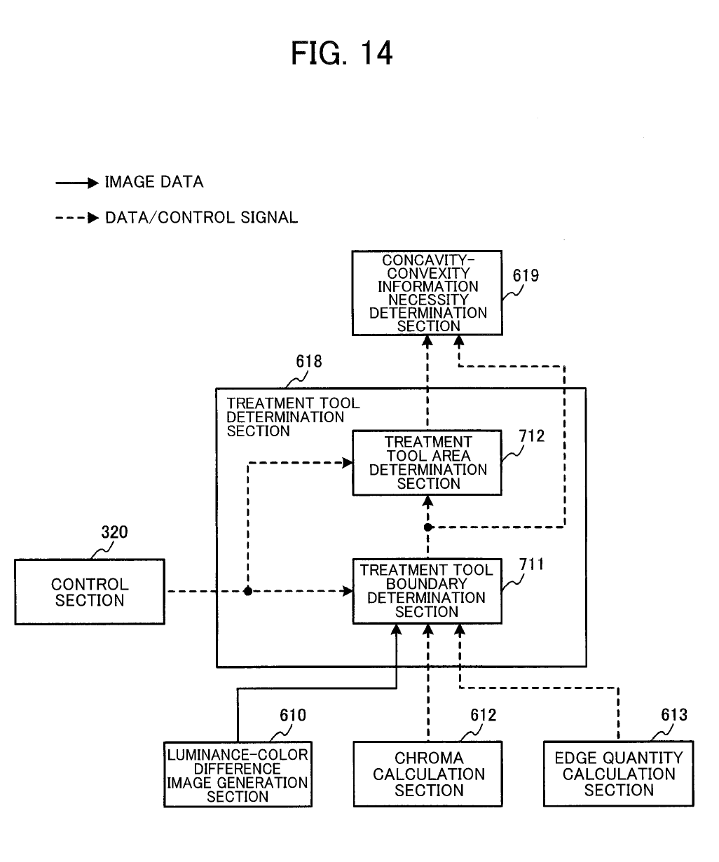

FIG. 14 illustrates a detailed configuration example of the treatment tool determination section 618. The treatment tool determination section 618 includes a treatment tool boundary determination section 711 and a treatment tool area determination section 712.

The chroma calculation section 612, the edge quantity calculation section 613, and the luminance-color difference image generation section 610 are connected to the treatment tool boundary determination section 711. The treatment tool boundary determination section 711 is connected to the treatment tool area determination section 712. The treatment tool area determination section 712 is connected to the concavity-convexity information necessity determination section 619. The control section 320 is connected to the treatment tool boundary determination section 711 and the treatment tool area determination section 712, and controls the treatment tool boundary determination section 711 and the treatment tool area determination section 712.

The treatment tool boundary determination section 711 determines a pixel of the reference image that corresponds to a treatment tool based on the chroma value S(x, y) output from the chroma calculation section 612 and the edge quantity E(x, y) output from the edge quantity calculation section 613, and outputs the determination results to the concavity-convexity information necessity determination section 619 using a determination signal. The determination signal is set to “1” corresponding to a pixel that has been determined to correspond to a treatment tool, and set to “0” corresponding to a pixel other than the pixel that has been determined to correspond to a treatment tool, for example.

A treatment tool has a large edge quantity E(x, y) and a small chroma value S(x, y) as compared with tissue. Therefore, a pixel for which the chroma value S(x, y) is smaller than a given threshold value th_S and the edge quantity E(x, y) is larger than a given threshold value th_E3, is determined to correspond to a treatment tool (see the following expression (18)).

$$\{ \frac{S ( x , y )}{Y ( x , y )} \} < th\_S and E ( x , y ) > th\_E 3 \qquad ( 18 )$$

The chroma value S(x, y) normally increases in proportion to the luminance value Y(x, y) irrespective of the color of the object. Therefore, the chroma value S(x, y) is normalized (divided) by the luminance value Y(x, y) (see the expression (18)).

A large edge quantity E(x, y) is observed only at the (treatment tool boundary) between a treatment tool and tissue, and the edge quantity E(x, y) is small in the inner area (treatment tool center area) of a treatment tool enclosed by the treatment tool boundary. Therefore, if a treatment tool is determined based only on the edge quantity E(x, y) and the chroma value S(x, y), only pixels that correspond to the treatment tool boundary are determined to be a treatment tool, and pixels that correspond to the treatment tool center area are not determined to be a treatment tool. According to the first embodiment, the treatment tool area determination section 712 determines pixels that correspond to the treatment tool center area to be a treatment tool.

Specifically, the treatment tool area determination section 712 determines pixels that correspond to the treatment tool center area to be a treatment tool using the method described above with reference to FIG. 12, and outputs the determination results to the concavity-convexity information necessity determination section 619 using a determination signal. The determination signal is set to “1” corresponding to a pixel that has been determined to correspond to a treatment tool, and set to “0” corresponding to a pixel other than the pixel that has been determined to correspond to a treatment tool, for example.

A given value may be set as the threshold values th_Y, th_dark, th_S, th_E1, th_E3, and co_NE in advance, or the user may set the threshold values th_Y, th_dark, th_S, th_E1, th_E3, and co_NE through the external I/F section 500.

The concavity-convexity information necessity determination section 619 determines the necessity of the extracted concavity-convexity information about each pixel based on the determination results output from the residue determination section 614, the bright spot determination section 615, the dark area determination section 616, the flat area determination section 617, and the treatment tool determination section 618, and outputs the necessity determination results to the concavity-convexity information correction section 316. Specifically, the concavity-convexity information necessity determination section 619 determines that the extracted concavity-convexity information about a pixel that has been determined to correspond to a residue, a bright spot, a dark area, a flat area, or a treatment tool (i.e., a pixel for which one of the determination signals is set to “1”) is “unnecessary” (i.e., the exclusion or reduction target). For example, the concavity-convexity information necessity determination section 619 sets the determination signal to “1” corresponding to a pixel for which it has been determined that the extracted concavity-convexity information is “unnecessary”, and outputs the determination signal as the determination results.

### 2.5. Concavity-Convexity Information Correction Process

The process performed by the concavity-convexity information correction section 316 is described in detail below. The concavity-convexity information correction section 316 performs a process that corrects the concavity-convexity map based on the necessity determination results (determination signal). Specifically, the concavity-convexity information correction section 316 performs a low-pass filtering process on a pixel on the concavity-convexity map that corresponds to a pixel for which it has been determined that the extracted concavity-convexity information is “unnecessary” (i.e., excluded or reduced) (e.g., a pixel for which the determination signal is set to “1”). The concavity-convexity information correction section 316 thus reduces the extracted concavity-convexity information about the pixel that has been determined to correspond to a residue, a bright spot, a dark area, a flat area, or a treatment tool. The concavity-convexity information correction section 316 outputs the concavity-convexity map subjected to the low-pass filtering process to the enhancement processing section 317.

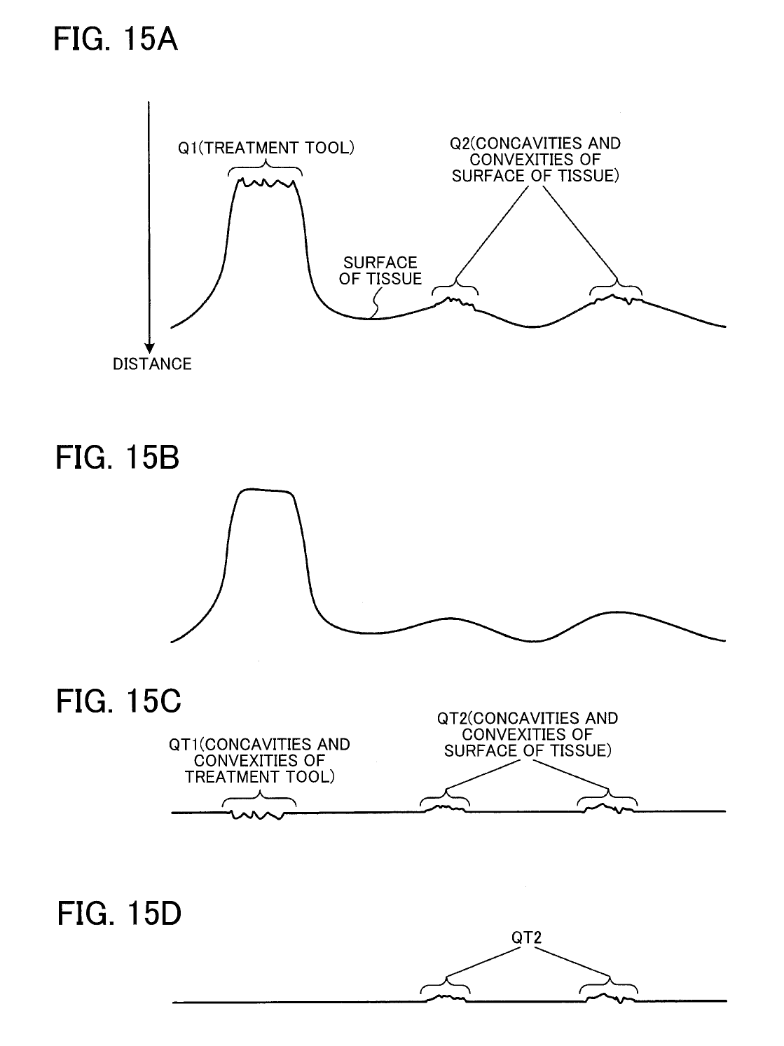

This process is described in detail below with reference to FIGS. 15A to 15D. FIG. 15A illustrates an example of the distance map. FIG. 15A illustrates the distance map represented by one-dimensional data for convenience of explanation. Q1 is an area in which a treatment tool is present, and Q2 is an area in which concavities and convexities are present on the surface of tissue. FIG. 15B illustrates an example of the results after the extraction section 603 has performed the low-pass filtering process on the distance map. The extraction section 603 subtracts the distance map subjected to the low-pass filtering process (FIG. 15B) from the original distance map (FIG. 15A) to generate a concavity-convexity map (FIG. 15C). Since the concavity-convexity map also includes concavity-convexity information QT1 about the treatment tool, the treatment tool area irrelevant to a diagnosis is also enhanced if the enhancement processing section 317 performs the enhancement process based on the concavity-convexity map, and an image that is difficult for the doctor to observe is generated.

According to the first embodiment, the determination section 315 determines a pixel that corresponds to a treatment tool, a residue, a bright spot, a dark area, or a flat area, and determines that the extracted concavity-convexity information about the pixel is “unnecessary”. The concavity-convexity information correction section 316 corrects the concavity-convexity map by performing the low-pass filtering process on the pixels on the distance map for which it has been determined that the extracted concavity-convexity information is “unnecessary” (FIG. 15D). This makes it possible to reduce the extracted concavity-convexity information about a pixel that corresponds to a treatment tool, a residue, a bright spot, a dark area, or a flat area. Since only concavity-convexity information QT2 about the surface of tissue remains in the concavity-convexity map, it is possible to enhance only the concavity-convexity structure of the surface of tissue without enhancing an area that is irrelevant to a diagnosis.

### 2.6. Enhancement Process

The process performed by the enhancement processing section 317 is described in detail below. An example in which the enhancement processing section 317 performs a process that enhances a given color component is described below. Note that the configuration is not limited thereto. The enhancement processing section 317 may perform various enhancement processes such as a contrast correction process.

The enhancement processing section 317 performs the enhancement process represented by the following expression (19). Note that diff(x, y) is the extracted concavity-convexity information calculated by the concavity-convexity information acquisition section 314 using the expression (1). As is clear from the expression (1), diff(x, y)>0 in an area (recess) deeper than the distance map subjected to the low-pass filtering process. R(x, y)′, G(x, y)′, and B(x, y)′ are respectively the R signal value, the G signal value, and the B signal value at the coordinates (x, y) after the enhancement process has been performed. The coefficients Co_R, Co_G, and Co_B are an arbitrary real number larger than 0. The coefficients Co_R, Co_G, and Co_B may be set to a given value in advance, or the user may set the coefficients Co_R, Co_G, and Co_B through the external I/F section 500.

if {diff(x,y)>0}

R(x,y)′=R(x,y)×{1−Co_{—}R×diff(x,y)}

G(x,y)′=G(x,y)×{1−Co_{—}G×diff(x,y)}

B(x,y)′=B(x,y)×{1+Co_{—}B×diff(x,y)}

else

R(x,y)′=R(x,y)

G(x,y)′=G(x,y)

B(x,y)′=B(x,y) (19)

Since the above enhancement process enhances the B signal value corresponding to a recess (diff(x, y)>0), it is possible to generate a display image in which the degree of blueness of the recess is enhanced. Since the degree of blueness is enhanced to a larger extent as the absolute value of the extracted concavity-convexity information diff (x, y) increases, the degree of blueness increases as the depth increases (i.e., the degree of blueness of a deeper area of the recess increases). This makes it possible to simulate a state in which a dye such as indigo carmine has been sprayed.

### 2.7. Modifications

Although an example in which the necessity of the concavity-convexity information is determined on a pixel basis has been described above, the configuration is not limited thereto. For example, the necessity of the concavity-convexity information may be determined on a local area (nxn) basis. The number of determinations and the circuit scale can be reduced by determining the necessity of the concavity-convexity information on a local area basis. If the size of the local area is increased to a large extent, a block-like artifact may occur in the image obtained by the enhancement process. Therefore, the size of the local area may be set so that an artifact does not occur.

Although an example in which a primary-color Bayer imaging method is used has been described above, the configuration is not limited thereto. Another imaging method (e.g. frame-sequential imaging method, complementary-color single-ship imaging method, primary-color two-chip imaging method, or primary-color three-chip imaging method) may also be used.

Although an example in which the observation mode is a normal light observation mode that utilizes a white light source has been described above, the configuration is not limited thereto. For example, a special light observation mode (e.g., narrow-band imaging (NBI)) may also be used. Note that a residue is observed in red during NBI, differing from normal light observation. Specifically, while the hue value of a residue during normal light observation is 270 to 310 (deg), the hue value of a residue during NBI is 0 to 20 and 340 to 359 (deg). Therefore, the residue determination section 614 determines a pixel having a hue value H(x, y) of 0 to 20 and 340 to 359 (deg) to be a residue, for example.

### 2.8. Software

Although an example in which each section included in the processor section 300 is implemented by hardware has been described above, the configuration is not limited thereto. For example, a CPU may perform the process of each section on image signals acquired using an imaging device, and the distance information. Specifically, the process of each section may be implemented by means of software by causing the CPU to execute a program. Alternatively, part of the process of each section may be implemented by means of software.

In this case, a program stored in an information storage device is read, and executed by a processor (e.g., CPU). The information storage device (computer-readable device) stores a program, data, and the like. The function of the information storage device may be implemented by an optical disk (e.g., DVD or CD), a hard disk drive (HDD), a memory (e.g., memory card or ROM), or the like. The processor (e.g., CPU) performs various processes according to the first embodiment based on the program (data) stored in the information storage device. Specifically, a program that causes a computer (i.e., a device that includes an operation section, a processing section, a storage section, and an output section) to function as each section according to the first embodiment (i.e., a program that causes a computer to execute the process implemented by each section) is stored in the information storage device.

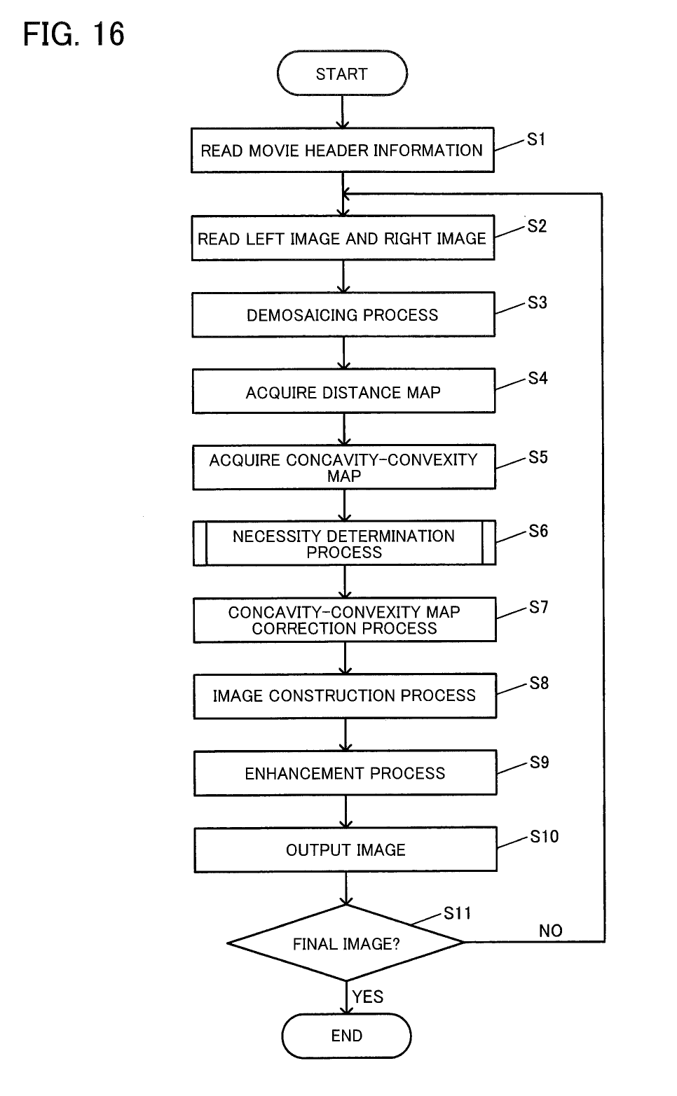

FIG. 16 is a flowchart when the process performed by the image processing section 310 is implemented by software. The header information relating to the imaging conditions is read (step S1). The header information includes the optical magnification of the imaging section 200 (with respect to the distance information), the distance between the image sensors 241 and 242, and the like.

The stereo image (left image and right image) acquired by the imaging section 200 is read (step S2). The demosaicing process is performed on the stereo image (step S3). The distance map (distance information) of the reference image (left image) is acquired based on the header information and the stereo image (subjected to the demosaicing process) using a stereo matching technique (step S4). The information about the concavity-convexity part of tissue is extracted from the distance map to acquire the concavity-convexity map (extracted concavity-convexity information) (step S5).

The necessity of the extracted concavity-convexity information about each pixel of the reference image (i.e., whether or not the extracted concavity-convexity information is excluded or reduced) is determined using the above method (step S6). The details of the flow of the necessity determination process are described later. The low-pass filtering process is performed on the extracted concavity-convexity information that corresponds to a pixel for which it has been determined in the step S6 that the extracted concavity-convexity information is “unnecessary” (excluded or reduced) to correct the concavity-convexity map (step S7). A known WB process, a known γ process, and the like are performed on the reference image (step S8). The process that enhances a concavity-convexity part using the expression (19) is performed on the reference image subjected to the step S8 based on the concavity-convexity map corrected in the step S7 (step S9), and the image subjected to the enhancement process is output (step S10).

The process is terminated when all of the images included in the movie have been processed. The step S2 is performed gain when all of the images have not been processed (step S11).

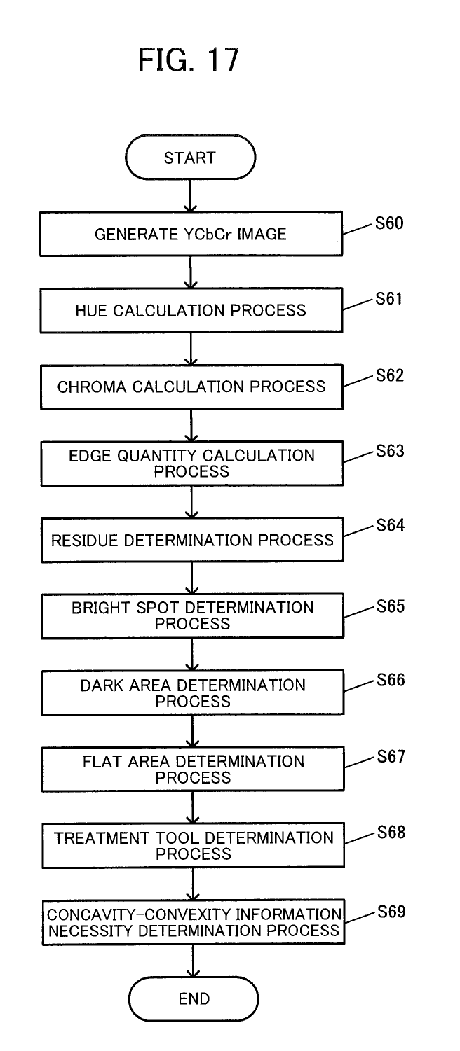

FIG. 17 is a flowchart illustrating the details of the necessity determination process performed in the step S6. The reference image (RGB image) is converted into a YCbCr image using the expression (4) (step S61).

The hue value H(x, y) of the reference image is calculated on a pixel basis using the expressions (5) to (11) (step S61). The chroma value S(x, y) of the reference image is calculated on a pixel basis using the expression (12) (step S62). The edge quantity E(x, y) of the reference image is calculated on a pixel basis using the expression (13) (step S63). Note that the steps S61 to S63 are performed in random order.

A pixel having a hue value H(x, y) of 270 to 310 (deg) is determined to correspond to a residue (step S64). A pixel for which the luminance value Y(x, y) and the edge quantity E(x, y) satisfy the expression (14), and a pixel situated within an area enclosed by such a pixel are determined to correspond to a bright spot (step S65). A pixel for which the luminance value Y(x, y) satisfies the expression (15) is determined to correspond to a dark area (step S66). A pixel for which the edge quantity E(x, y) satisfies the expression (16) is determined to correspond to a flat area (step S67). A pixel for which the chroma value S(x, y) and the edge quantity E(x, y) satisfy the expression (18), and a pixel situated within an area enclosed by such a pixel are determined to correspond to a treatment tool (step S68). Note that the steps S64 to S68 are performed in random order.

The extracted concavity-convexity information about a pixel that has been determined to correspond to a residue, a bright spot, a dark area, a flat area, or a treatment tool (steps S64 to S68) is determined to be “unnecessary” (excluded or reduced) (step S69).

According to the first embodiment, since only a concavity-convexity part in the surface area of tissue can be enhanced without spraying a dye, it is possible to reduce the burden imposed on the doctor and the patient. Since an area (e.g., residue or treatment tool) that is unnecessary for a diagnosis is not enhanced, it is possible to provide an image that is easy for the doctor to observe. Since it is possible to suppress a situation in which an area where concavities and convexities are not present (e.g., flat area, dark area, or bright spot area) is enhanced, it is possible to reduce the risk of a misdiagnosis. Since it is unnecessary to provide a range sensor (described later in connection with the second embodiment), it is possible to relatively simplify the configuration of the imaging section 200.

According to the first embodiment, the determination section 315 determines whether or not the feature quantity based on the pixel value of the captured image satisfies a given condition that corresponds to the exclusion or reduction target corresponding to each given area (a pixel or a block having a given size).

It is possible to determine the concavity-convexity information of an object that is not useful for the subsequent process by setting a condition relating to the feature quantity of the target for which the concavity-convexity information is excluded or reduced as the given condition, and detecting an area that satisfies the given condition.

According to the first embodiment, the determination section 315 determines to exclude or reduce the extracted concavity-convexity information about a given area (e.g., pixel) for which the hue value H(x, y) satisfies a given condition. For example, the given condition is a condition whereby the hue value H(x, y) belongs to a given range (e.g., 270 to 310 (deg)) that corresponds to the color of a residue.

It is possible to determine an area that satisfies a hue condition to be an object that is not useful for the subsequent process by setting the hue specific to the exclusion or reduction target (e.g., residue) as the given condition.

According to the first embodiment, the determination section 315 determines to exclude or reduce the extracted concavity-convexity information about a given area for which the chroma value S(x, y) satisfies a given condition. For example, the given condition is a condition whereby the chroma value S(x, y) belongs to a given range that corresponds to the color of a treatment tool. More specifically, the given condition is a condition whereby a value obtained by dividing the chroma value S(x, y) by the luminance value Y(x, y) is smaller than the chroma threshold value th_S that corresponds to the chroma of a treatment tool, and the edge quantity E(x, y) is larger than the edge quantity threshold value th_E3 that corresponds to the edge quantity of a treatment tool (expression (18)).

It is possible to determine an area that satisfies a chroma condition to be an object that is not useful for the subsequent process by setting the chroma specific to the exclusion or reduction target (e.g., treatment tool) as the given condition. Since a treatment tool has low chroma and a large edge quantity, a treatment tool area can be determined with high accuracy by combining chroma with edge quantity.

According to the first embodiment, the determination section 315 determines to exclude or reduce the extracted concavity-convexity information about a given area for which the luminance value Y(x, y) satisfies a given condition. For example, the given condition is a condition whereby the luminance value Y(x, y) is larger than the luminance threshold th_Y that corresponds to the luminance of a bright spot. More specifically, the given condition is a condition whereby the luminance value Y(x, y) is larger than the luminance threshold th_Y, and the edge quantity E(x, y) is larger than the edge quantity threshold value th_E1 that corresponds to the edge quantity of a bright spot (expression (14)). Alternatively, the given condition is a condition whereby the luminance value Y(x, y) is smaller than the luminance threshold th_dark that corresponds to the luminance of a dark area (expression (15)).

It is possible to determine an area that satisfies a luminance condition to be an object that is not useful for the subsequent process by setting the luminance (brightness) specific to the exclusion or reduction target (e.g., bright spot or dark area) as the given condition. Since a bright spot has high luminance and a large edge quantity, a bright spot area can be determined with high accuracy by combining luminance with edge quantity.

According to the first embodiment, the determination section 315 determines to exclude or reduce the extracted concavity-convexity information about a given area for which the edge quantity E(x, y) satisfies a given condition. For example, the given condition is a condition whereby the edge quantity E(x, y) is larger than the edge quantity threshold value th_E3 that corresponds to the edge quantity of a treatment tool (expression (18)). Alternatively, the given condition is a condition whereby the edge quantity E(x, y) is larger than the edge quantity threshold value th_E1 that corresponds to the edge quantity of a bright spot (expression (14)). Alternatively, the given condition is a condition whereby the edge quantity E(x, y) is smaller than the edge quantity threshold value th_E2(x, y) that corresponds to the edge quantity of a flat area (expression (16)).

It is possible to determine an area that satisfies an edge quantity condition to be an object that is not useful for the subsequent process by setting the edge quantity (e.g., a high-frequency component of the image or the pixel value of a differential image) specific to the exclusion or reduction target (e.g., flat area) as the given condition.

According to the first embodiment, the determination section 315 increases the edge quantity threshold value th_E2(x, y) as the luminance value Y(x, y) increases corresponding to the noise characteristics noise{Y(x, y)} of the captured image in which the amount of noise increases as the luminance value Y(x, y) increases (expression (17)).

Since a change in pixel value due to concavities and convexities of the object is small in a flat area, a change in pixel value due to noise affects the edge quantity. Therefore, a flat area can be determined with high accuracy without being affected by the amount of noise by setting the edge quantity threshold value corresponding to the amount of noise.

According to the first embodiment, the image acquisition section 350 (demosaicing section 311) acquires a stereo image (parallax image) as the captured image. The distance information acquisition section 313 acquires the distance information (e.g., distance map) by performing the stereo matching process on the stereo image. The determination section 315 determines to exclude or reduce the extracted concavity-convexity information about a given area for which the feature quantity based on the captured image satisfies a given condition that corresponds to a bright spot, a dark area, and a flat area.

Since a bright spot occurs due to specular reflection from the surface of a mucous membrane, a bright spot occurs at a position that differs between the left image and the right image that differ in viewpoint. Therefore, wrong distance information may be detected in a bright spot area by stereo matching. Since noise is predominant in a dark area, the stereo matching accuracy may deteriorate due to noise. Since a change in pixel value due to concavities and convexities of the object is small in a flat area, the stereo matching accuracy may deteriorate due to noise. According to the first embodiment, since it is possible to detect a bright spot, a dark area, and a flat area, it is possible to exclude or reduce the extracted concavity-convexity information generated from wrong distance information.

### 3. Second Embodiment

### 3.1. Endoscope Apparatus

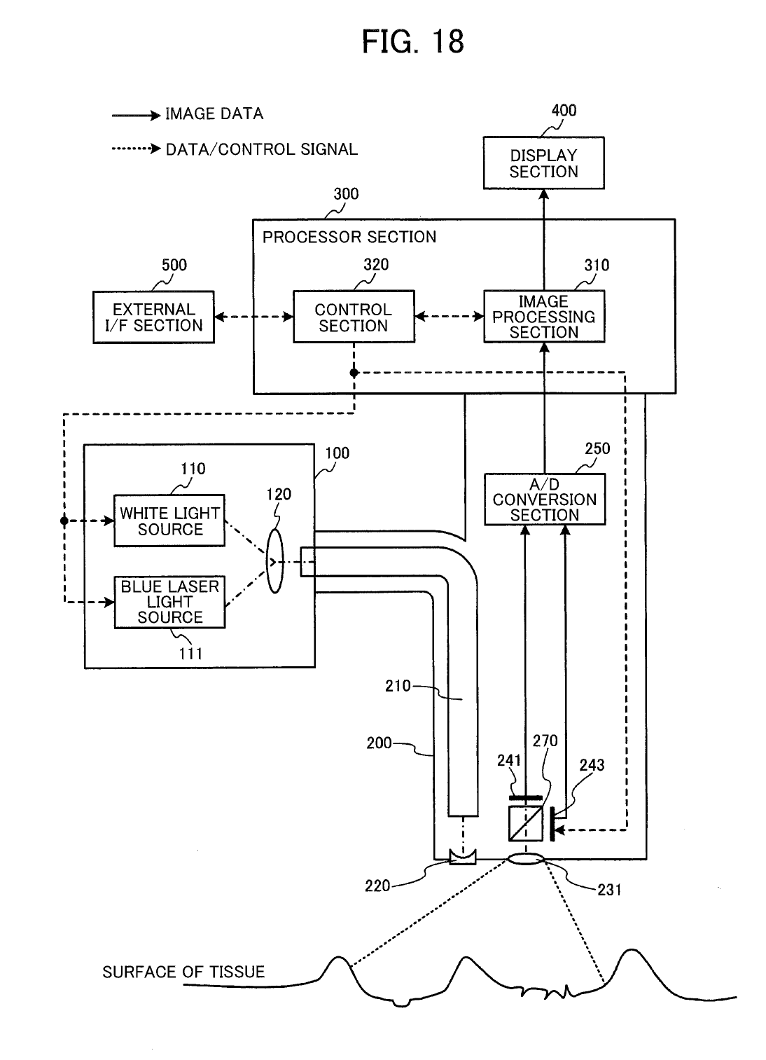

FIG. 18 illustrates a configuration example of an endoscope apparatus according to a second embodiment of the invention. The endoscope apparatus includes a light source section 100, an imaging section 200, a processor section 300 (control device), a display section 400, and an external IN section 500. Note that the configuration of the display section 400 and the external I/F section 500 is the same as described above in connection with the first embodiment, and description thereof is omitted. The following description focuses on the differences in configuration and operation from the first embodiment, and description of the same configuration and operation as those described above in connection with the first embodiment is appropriately omitted.

The light source section 100 includes a white light source 110, a blue laser light source 111, and a condenser lens 120 that focuses light obtained by synthesizing light emitted from the white light source 110 and light emitted from the blue laser light source 111 on a light guide fiber 210.

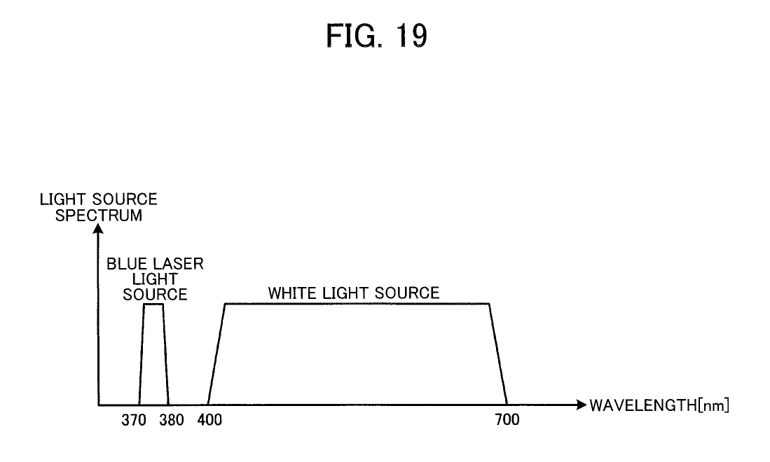

The white light source 110 and the blue laser light source 111 are controlled in a pulsed manner based on a control signal output from the control section 320. As illustrated in FIG. 19, the white light source 110 has a spectral band of 400 to 700 nm, and the blue laser light source 111 has a spectral band of 370 to 380 nm.

The imaging section 200 includes the light guide fiber 210, an illumination lens 220, an objective lens 231, an image sensor 241, a range sensor 243, an A/D conversion section 250, and a dichroic prism 270. Note that the light guide fiber 210, the illumination lens 220, the objective lens 231, and the image sensor 241 are configured in the same manner as described above in connection with the first embodiment, and description thereof is omitted.

The dichroic prism 270 reflects short-wavelength light having a wavelength of 370 to 380 nm (that corresponds to the spectral band of the blue laser light source 111), and allows light having a wavelength of 400 to 700 nm (that corresponds to the spectral band of the white light source 110) to pass through. The short-wavelength light (emitted from the blue laser light source 111) reflected by the dichroic prism 270 is detected by the range sensor 243. The light that has passed through the dichroic prism 270 (i.e., the light emitted from the white light source 110, and reflected by the object) forms an image on the image sensor 241. The range sensor 243 is a Time-of-Flight (TOF) range sensor that measures distance based on the time from the blue laser light emission start timing to the reflected light (reflected blue laser light) detection timing. Information about the blue laser light emission start timing is transmitted from the control section 320.

The A/D conversion section 250 converts analog signals (distance information) acquired by the range sensor 243 into digital signals (distance information (distance map)), and outputs the digital signals to the image processing section 310.

The processor section 300 includes an image processing section 310 and the control section 320. The image processing section 310 performs image processing (described later) on the image output from the A/D conversion section 250 to generate a display image, and outputs the generated display image to the display section 400. The control section 320 controls the operation of the image processing section 310 based on a signal output from the external I/F section 500. The control section 320 is connected to the white light source 110, the blue laser light source 111, and the range sensor 243, and controls the white light source 110, the blue laser light source 111, and the range sensor 243.

### 3.2. Image Processing Section

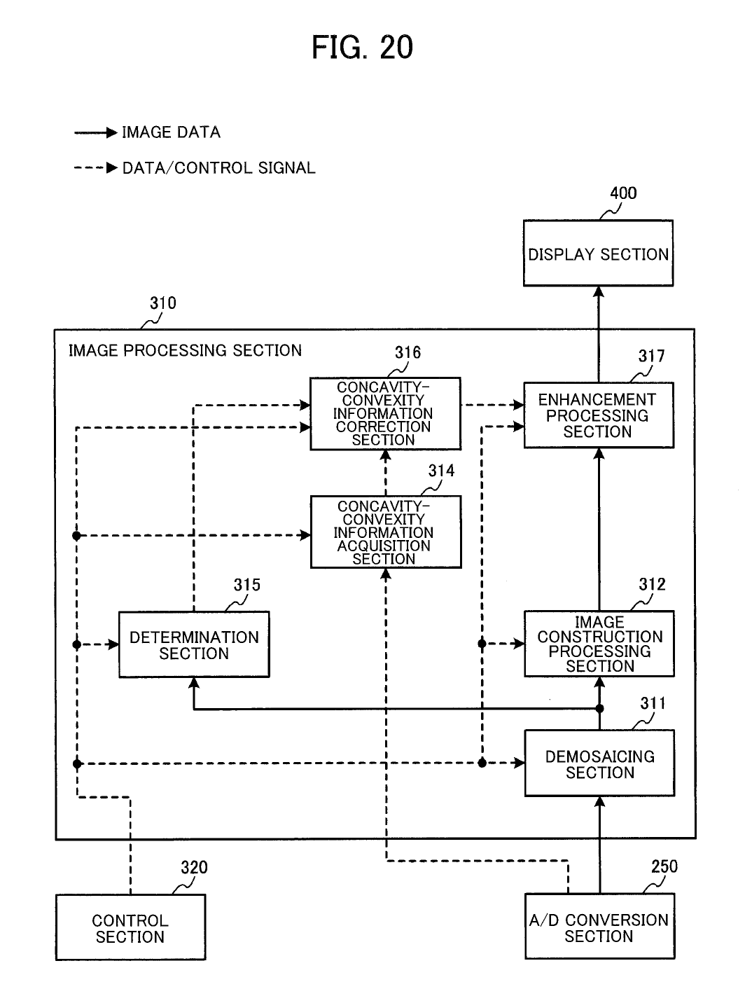

FIG. 20 illustrates a detailed configuration example of the image processing section 310. The image processing section 310 includes a demosaicing section 311, an image construction processing section 312, a concavity-convexity information acquisition section 314, a determination section 315, a concavity-convexity information correction section 316, and an enhancement processing section 317. Note that the configuration of the demosaicing section 311, the image construction processing section 312, and the enhancement processing section 317 is the same as described above in connection with the first embodiment, and description thereof is omitted. In the second embodiment, the A/D conversion section 250 (or a readout section (not illustrated in the drawings) that reads the distance map from the A/D conversion section 250) corresponds to the distance information acquisition section 313 illustrated in FIG. 1.

The A/D conversion section 250 is connected to the demosaicing section 311 and the concavity-convexity information acquisition section 314. The demosaicing section 311 is connected to the image construction processing section 312 and the determination section 315. The determination section 315 and the concavity-convexity information acquisition section 314 are connected to the concavity-convexity information correction section 316. The concavity-convexity information correction section 316 and the image construction processing section 312 are connected to the enhancement processing section 317. The enhancement processing section 317 is connected to the display section 400. The control section 320 is connected to the demosaicing section 311, the image construction processing section 312, the concavity-convexity information acquisition section 314, the determination section 315, the concavity-convexity information correction section 316, and the enhancement processing section 317, and controls the demosaicing section 311, the image construction processing section 312, the concavity-convexity information acquisition section 314, the determination section 315, the concavity-convexity information correction section 316, and the enhancement processing section 317.

The concavity-convexity information extraction section 314 calculates concavity-convexity information about the surface of tissue (excluding the distance information that depends on the shape of the digestive tract (e.g., lumen and folds)) as a concavity-convexity map (extracted concavity-convexity information) from the distance information output from the A/D conversion section 250. The concavity-convexity map is calculated in the same manner as described above in connection with the first embodiment.

When the distance map is acquired using the range sensor 243, it is possible to acquire an accurate concavity-convexity map corresponding to a bright spot, a dark area, and a flat area. Therefore, it is possible to solve the problem specific to stereo matching (i.e., the stereo matching accuracy deteriorates in a bright spot, a dark area, and a flat area) described above in connection with the first embodiment.

Therefore, it is unnecessary to determine a bright spot, a dark area, and a flat area. However, a problem in which a residue or a treatment tool is enhanced also occurs when using the method according to the second embodiment. Specifically, the concavity-convexity information about an area (e.g., residue or treatment tool) that is irrelevant to a diagnosis is enhanced. Therefore, a process that determines a residue and a treatment tool is performed in the second embodiment.

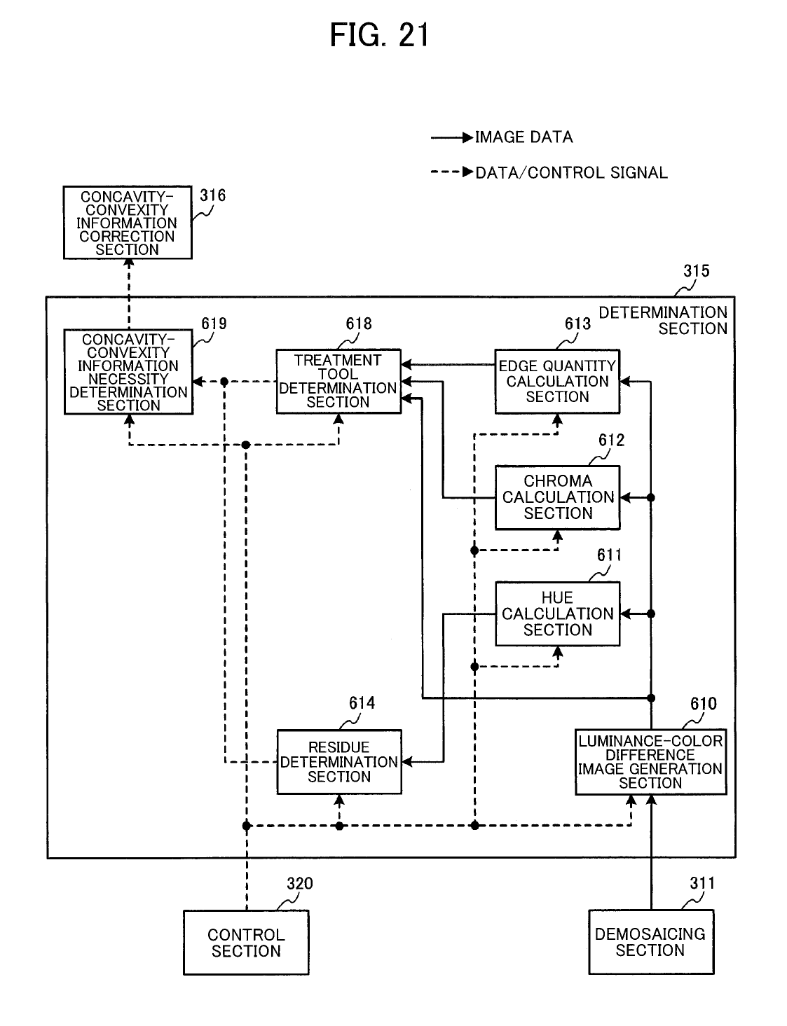

FIG. 21 illustrates a detailed configuration example of the determination section 315. The determination section 315 includes a luminance-color difference image generation section 610, a hue calculation section 611, a chroma calculation section 612, an edge quantity calculation section 613, a residue determination section 614, a treatment tool determination section 618, and a concavity-convexity information necessity determination section 619.

The determination section 315 according to the second embodiment differs from the determination section 315 according to the first embodiment in that the determination section 315 according to the second embodiment does not include the bright spot determination section 615, the dark area determination section 616, and the flat area determination section 617. The residue determination section 614 determines a pixel that corresponds to a residue based on the hue value H(x, y), and the treatment tool determination section 618 determines a pixel that corresponds to a treatment tool based on the edge quantity E(x, y), the chroma value S(x, y), and the luminance value Y(x, y). The concavity-convexity information necessity determination section 619 determines to exclude or reduce the extracted concavity-convexity information about a pixel that has been determined to correspond to a residue or a treatment tool. The details of the process performed by each section are the same as described above in connection with the first embodiment, and description thereof is omitted.

According to the second embodiment, since only a concavity-convexity part in the surface area of tissue can be enhanced without spraying a dye, it is possible to reduce the burden imposed on the doctor and the patient. Since an area (e.g., residue or treatment tool) that is unnecessary for a diagnosis is not enhanced, it is possible to provide an image that is easy for the doctor to observe. Since the distance map is acquired using the range sensor 243, it is unnecessary to determine a bright spot, a dark area, and a flat area. Therefore, the circuit scale of the processor can be reduced as compared with the first embodiment.

Although an example in which the A/D conversion section 250 acquires the distance map has been described above, the configuration is not limited thereto. For example, the image processing section 310 may include a distance information acquisition section 313, and the distance information acquisition section 313 may calculate a defocus parameter from the captured image, and acquire the distance information based on the defocus parameter. In this case, the distance information acquisition section 313 acquires a first image and a second image while shifting the focus lens position, converts each image into a luminance value, calculates a second derivative of the luminance value of each image, and calculates the average value thereof. The distance information acquisition section 313 calculates the difference between the luminance value of the first image and the luminance value of the second image, divides the difference by the average second derivative value, calculates the defocus parameter, and acquires the distance information from the relationship between the defocus parameter and the object distance (e.g., stored in a look-up table). Note that the blue laser light source 111 and the range sensor 243 may be omitted when using this method.

### 3.3. Software

Although an example in which each section included in the processor section 300 is implemented by hardware has been described above, the configuration is not limited thereto. For example, a CPU may perform the process of each section on image signals acquired using an imaging device, and the distance information. Specifically, the process of each section may be implemented by means of software by causing the CPU to execute a program. Alternatively, part of the process of each section may be implemented by means of software.

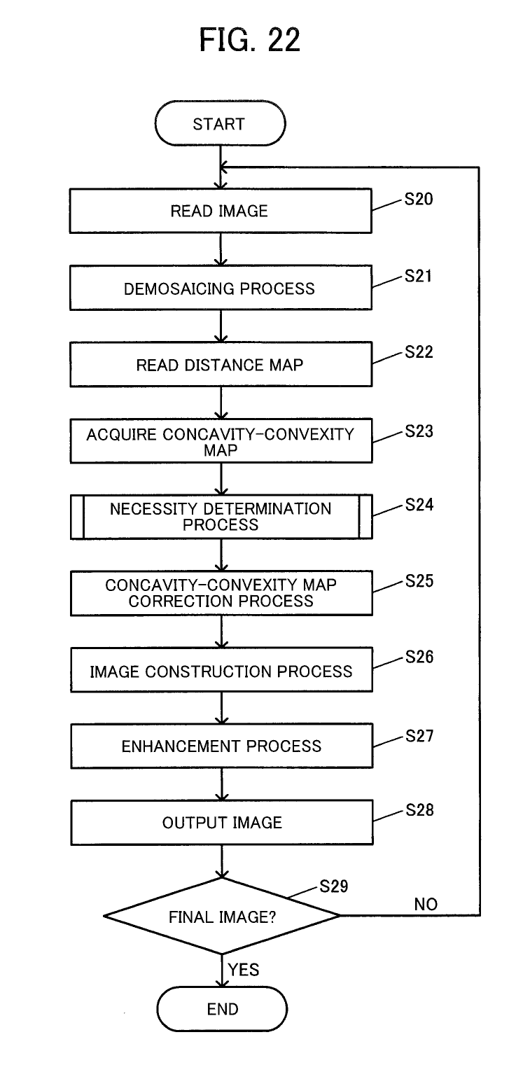

FIG. 22 is a flowchart when the process performed by the image processing section 310 is implemented by software. The image acquired by the imaging section 200 is read (step S20).

The demosaicing process is performed on the image (step S21). The distance map (distance information) acquired by the range sensor 243 is read (step S22). The information about the concavity-convexity part of tissue is extracted from the distance map to acquire the concavity-convexity map (extracted concavity-convexity information) (step S23).

The necessity of the extracted concavity-convexity information about each pixel of the captured image (i.e., whether or not the extracted concavity-convexity information is excluded or reduced) is determined using the above method (step S24). The details of the flow of the necessity determination process are described later. The low-pass filtering process is performed on the extracted concavity-convexity information that corresponds to a pixel for which it has been determined in the step S24 that the extracted concavity-convexity information is “unnecessary” (excluded or reduced) to correct the concavity-convexity map (step S25). A known WB process, a known γ process, and the like are performed on the captured image (step S26). The process that enhances a concavity-convexity part using the expression (19) is performed on the captured image subjected to the step S26 based on the concavity-convexity map corrected in the step S25 (step S27), and the image subjected to the enhancement process is output (step S28).

The process is terminated when all of the images included in the movie have been processed. The step S20 is performed again when all of the images have not been processed (step S29).

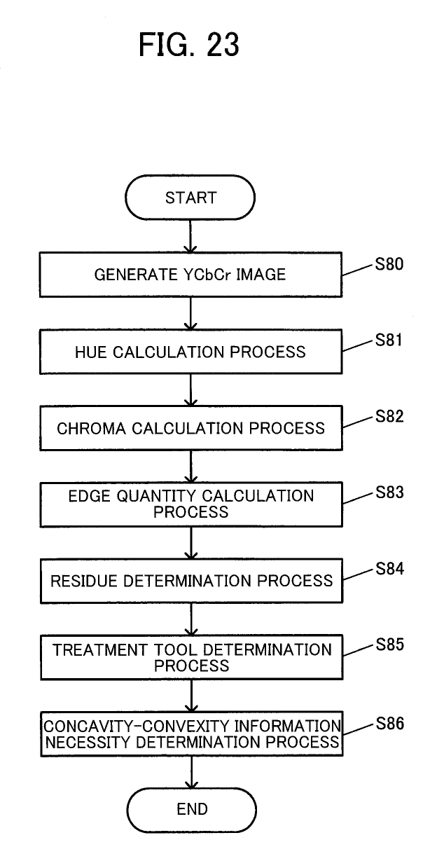

FIG. 23 is a flowchart illustrating the details of the necessity determination process performed in the step S24. The steps S80 to S86 illustrated in FIG. 23 correspond to the steps S60 to S64, S68, and S69 illustrated in FIG. 17 (see the first embodiment). Specifically, the steps S65 to S67 are omitted from the flow according to the first embodiment. The process performed in each step is the same as described above in connection with the first embodiment, and description thereof is omitted.

According to the second embodiment, the distance information acquisition section (e.g., the A/D conversion section 250, or a readout section (not illustrated in the drawings) that reads the distance information from the A/D conversion section 250) acquires the distance information (e.g., distance map) based on a ranging signal from the range sensor 243 (e.g., TOF range sensor) included in the imaging section 200. The determination section 315 determines to exclude or reduce the extracted concavity-convexity information about a given area for which the feature quantity based on the captured image satisfies a given condition that corresponds to a treatment tool and a residue.

According to the second embodiment, since the distance information can be acquired using the range sensor 243, erroneous detection by stereo matching does not occur, differing from the case of acquiring the distance information from a stereo image. Therefore, since it is unnecessary to determine a bright spot, a dark area, and a flat area, and the necessity determination process can be simplified, the circuit scale and the amount of processing can be reduced.

The image processing device (image processing section 310) and the like according to the second embodiment may include a processor and a memory. The processor may be a central processing unit (CPU), for example. Note that the processor is not limited to a CPU. Various other processors such as a graphics processing unit (GPU) or a digital signal processor (DSP) may also be used. The processor may be a hardware circuit that includes an ASIC. The memory stores a computer-readable instruction. Each section of the image processing device (image processing section 310) and the like according to the second embodiment is implemented by causing the processor to execute the instruction. The memory may be a semiconductor memory (e.g., SRAM or DRAM), a register, a hard disk, or the like. The instruction may be an instruction included in an instruction set that is included in a program, or may be an instruction that causes a hardware circuit included in the processor to operate.

Although only some embodiments of the invention and the modifications thereof have been described in detail above, those skilled in the art will readily appreciate that many modifications are possible in the embodiments and the modifications thereof without materially departing from the novel teachings and advantages of the invention. A plurality of elements described in connection with the above embodiments and the modifications thereof may be appropriately combined to implement various configurations. For example, some of the elements described in connection with the above embodiments and the modifications thereof may be omitted. Some of the elements described in connection with different embodiments and modifications thereof may be appropriately combined. Specifically, various modifications and applications are possible without materially departing from the novel teachings and advantages of the invention. Any term cited with a different term having a broader meaning or the same meaning at least once in the specification and the drawings can be replaced by the different term in any place in the specification and the drawings.

What is claimed is:

1. An image processing device comprising:
an image acquisition section that acquires a captured image that includes an image of an object;
a distance information acquisition section that acquires distance information based on a distance from an imaging section to the object when the imaging section captured the captured image;
a concavity-convexity information acquisition section that acquires concavity-convexity information about the object based on the distance information as extracted concavity-convexity information;
a determination section that determines whether or not to exclude or reduce the extracted concavity-convexity information corresponding to each given area of the captured image; and
a concavity-convexity information correction section that excludes the extracted concavity-convexity information corresponding to the given area for which the determination section has determined to exclude the extracted concavity-convexity information, or reduces a degree of concavities and convexities represented by the extracted concavity-convexity information corresponding to the given area for which the determination section has determined to reduce the extracted concavity-convexity information,
the concavity-convexity information acquisition section excluding a structure that is more global than a desired concavity-convexity part from the distance information based on known characteristic information to extract information about the desired concavity-convexity part as the extracted concavity-convexity information, the known characteristic information being information that represents known characteristics relating to a structure of the object.

2. The image processing device as defined in claim 1,
the determination section determining whether or not a feature quantity based on a pixel value of the captured image satisfies a given condition corresponding to each given area, the given condition being a condition that corresponds to an exclusion or reduction target for which the extracted concavity-convexity information is excluded or reduced.

3. The image processing device as defined in claim 2,
the determination section including a hue calculation section that calculates a hue value of the captured image as the feature quantity, and
the determination section determining to exclude or reduce the extracted concavity-convexity information about the given area for which the hue value satisfies the given condition.

4. The image processing device as defined in claim 3,
the given condition being a condition whereby the hue value belongs to a given range that corresponds to a color of a residue.

5. The image processing device as defined in claim 2,
the determination section including a chroma calculation section that calculates a chroma value of the captured image as the feature quantity, and
the determination section determining to exclude or reduce the extracted concavity-convexity information about the given area for which the chroma value satisfies the given condition.

6. The image processing device as defined in claim 5,
the given condition being a condition whereby the chroma value belongs to a given range that corresponds to a color of a treatment tool.

7. The image processing device as defined in claim 6,
the determination section including:
an edge quantity calculation section that calculates an edge quantity of the captured image as the feature quantity; and
a luminance calculation section that calculates a luminance value of the captured image as the feature quantity, and
the given condition being a condition whereby a value obtained by dividing the chroma value by the luminance value is smaller than a chroma threshold value that corresponds to chroma of the treatment tool, and the edge quantity is larger than an edge quantity threshold value that corresponds to the edge quantity of the treatment tool.

8. The image processing device as defined in claim 2,
the determination section including a luminance calculation section that calculates a luminance value of the captured image as the feature quantity, and
the determination section determining to exclude or reduce the extracted concavity-convexity information about the given area for which the luminance value satisfies the given condition.

9. The image processing device as defined in claim 8,
the given condition being a condition whereby the luminance value is larger than a luminance threshold that corresponds to luminance of a bright spot.

10. The image processing device as defined in claim 9,
the determination section including an edge quantity calculation section that calculates an edge quantity of the captured image as the feature quantity, and
the given condition being a condition whereby the luminance value is larger than the luminance threshold value, and the edge quantity is larger than an edge quantity threshold value that corresponds to the edge quantity of the bright spot.

11. The image processing device as defined in claim 8,
the given condition being a condition whereby the luminance value is smaller than a luminance threshold that corresponds to luminance of a dark area.

12. The image processing device as defined in claim 2,
the determination section including an edge quantity calculation section that calculates an edge quantity of the captured image as the feature quantity, and
the determination section determining to exclude or reduce the extracted concavity-convexity information about the given area for which the edge quantity satisfies the given condition.

13. The image processing device as defined in claim 12,
the given condition being a condition whereby the edge quantity is larger than an edge quantity threshold that corresponds to the edge quantity of a treatment tool.

14. The image processing device as defined in claim 12,
the given condition being a condition whereby the edge quantity is larger than an edge quantity threshold that corresponds to the edge quantity of a bright spot.

15. The image processing device as defined in claim 12,
the given condition being a condition whereby the edge quantity is smaller than an edge quantity threshold that corresponds to the edge quantity of a flat area.

16. The image processing device as defined in claim 15,
the determination section including a luminance calculation section that calculates a luminance value of the captured image as the feature quantity, and
the determination section increasing the edge quantity threshold value as the luminance value increases corresponding to noise characteristics of the captured image in which an amount of noise increases as the luminance value increases.

17. The image processing device as defined in claim 1,
the concavity-convexity information correction section performing a smoothing process on the extracted concavity-convexity information about the given area for which the determination section has determined to exclude or reduce the extracted concavity-convexity information.

18. The image processing device as defined in claim 1,
the concavity-convexity information correction section setting the extracted concavity-convexity information about the given area for which the determination section has determined to exclude or reduce the extracted concavity-convexity information, to a given value that corresponds to a non-concavity-convexity part.

19. The image processing device as defined in claim 1, further comprising:
an enhancement processing section that performs an enhancement process on the captured image based on the extracted concavity-convexity information output from the concavity-convexity information correction section.

20. The image processing device as defined in claim 1,
the concavity-convexity information acquisition section extracting a concavity-convexity part of the object that agrees with the characteristics specified by the known characteristic information from the distance information as the extracted concavity-convexity information based on the distance information and the known characteristic information.

21. The image processing device as defined in claim 1,
the image acquisition section acquiring a stereo image as the captured image,
the distance information acquisition section acquiring the distance information by performing a stereo matching process on the stereo image, and
the determination section determining to exclude or reduce the extracted concavity-convexity information about the given area for which a feature quantity based on the captured image satisfies a given condition that corresponds to a bright spot, a dark area, and a flat area.

22. The image processing device as defined in claim 1,
the distance information acquisition section acquiring the distance information based on a ranging signal output from a range sensor included in the imaging section, and
the determination section determining to exclude or reduce the extracted concavity-convexity information about the given area for which a feature quantity based on the captured image satisfies a given condition that corresponds to a treatment tool and a residue.

23. An endoscope apparatus comprising the image processing device as defined in claim 1.

24. An image processing method comprising:
acquiring a captured image that includes an image of an object;
acquiring distance information based on a distance from an imaging section to the object when the imaging section captured the captured image;
excluding a structure that is more global than a desired concavity-convexity part from the distance information based on known characteristic information to extract information about the desired concavity-convexity part as extracted concavity-convexity information, the known characteristic information being information that represents known characteristics relating to a structure of the object, and the extracted concavity-convexity information being concavity-convexity information about the object based on the distance information;
determining whether or not to exclude or reduce the extracted concavity-convexity information corresponding to each given area of the captured image; and
excluding the extracted concavity-convexity information corresponding to the given area for which it has been determined to exclude the extracted concavity-convexity information, or reducing a degree of concavities and convexities represented by the extracted concavity-convexity information corresponding to the given area for which it has been determined to reduce the extracted concavity-convexity information.

25. A non-transitory information storage device storing an image processing program that causes a computer to perform steps of:
acquiring a captured image that includes an image of an object;
acquiring distance information based on a distance from an imaging section to the object when the imaging section captured the captured image;
excluding a structure that is more global than a desired concavity-convexity part from the distance information based on known characteristic information to extract information about the desired concavity-convexity part as extracted concavity-convexity information, the known characteristic information being information that represents known characteristics relating to a structure of the object, and the extracted concavity-convexity information being concavity-convexity information about the object based on the distance information;
determining whether or not to exclude or reduce the extracted concavity-convexity information corresponding to each given area of the captured image; and
excluding the extracted concavity-convexity information corresponding to the given area for which it has been determined to exclude the extracted concavity-convexity information, or reducing a degree of concavities and convexities represented by the extracted concavity-convexity information corresponding to the given area for which it has been determined to reduce the extracted concavity-convexity information.
